(19) United States
(12) Patent Application Publication
(10) Pub. No.: US 2014/0012005 A1
(43) Pub. Date: Jan. 9, 2014

(54) PROCESS FOR PREPARING CHIRAL AMINO ACIDS

(71) Applicants: Sylvain Darses, Paris (FR); Nicolas Lefevre, Sartrouville (FR); Benoit Folleas, Senlis (FR); Jean-Louis Brayer, Nanteuille Haudouin (FR)

(72) Inventors: Sylvain Darses, Paris (FR); Nicolas Lefevre, Sartrouville (FR); Benoit Folleas, Senlis (FR); Jean-Louis Brayer, Nanteuille Haudouin (FR)

(73) Assignee: Diverchim, Montateire Cedex (FR)

(21) Appl. No.: 13/997,602

(22) PCT Filed: Dec. 22, 2011
(86) PCT No.: PCT/FR2011/053147

§ 371 Date: Sep. 24, 2013

(30) Foreign Application Priority Data
Dec. 23, 2010 (FR) 10/61229

(51) Int. Cl. C07C 269/04; C07D 333/24; C07D 307/54; C07D 401/04; C07D 333/60; C07D 307/91; C07F 7/10; C07C 315/04; C07D 317/46; C07D 209/08

(52) U.S. Cl. CPC C07C 269/04; C07D 317/46; C07D 333/24; C07D 307/54; C07D 209/08; C07D 333/60; C07D 307/91; C07F 7/10; C07C 315/04; C07D 401/04
USPC 546/193; 560/24; 560/32; 560/25; 549/441; 549/76; 549/493; 548/507; 549/58; 560/28; 549/461; 556/420; 560/11

(57) ABSTRACT

The present invention relates to a process for preparing chiral amino acids with excellent enantiomeric excesses.

The present invention relates to a process for preparing chiral amino acids with excellent enantiomeric excesses.

Among the building blocks of biologically active molecules, the chiral amino acids number among the most significant. They are therefore targets of choice for the pharmaceutical industry, with potential that increases as the enantiomeric excesses approach 100%. For good performance, the synthesis must therefore take place with the best possible control of the chiral centre.

The enzymatic resolution of racemic mixtures is possible. Moreover, several synthesis methods for chiral amino acids are available: thus, they can be prepared by Strecker and Ugi condensations or the functionalization of anions derived from glycine catalysed by a chiral phase-transfer agent (C. Nájera, J. M. Sansano, Chem. Rev. 2007, 107, 4584-4671). However, the route most used in industry is asymmetric hydrogenation of esters derived from dehydroalanine (M. J. Burk, M. F. Gross, J. P. Martinez, J. Am. Chem. Soc. 1995, 117, 9375-9376).

There are only a few examples describing enantioselective conjugated additions for α-aminoacrylates, in the presence of a chiral substance in a catalytic quantity, with the aim of obtaining a chiral α-amino acid.
Sibi et al. describe the radical conjugated addition of halogenated derivatives catalysed by chiral Lewis acids. (Sibi, M. P.; Asano, Y.; Sausker, J. B. Angew. Chem. Int. Ed. 2001, 40, 1293). The enantiomeric excesses do not exceed 85%.
The other cases all describe an addition reaction of an organoboron compound with an α-aminoacrylate followed by an enantioselective protonation. The reaction is catalysed by a rhodium/biphosphorus ligand combination. The organoboron compound is always selected from the potassium aryltrifluoroborates or arylboronic acids. However, the pH of the medium varies as well as the nature of the proton donor. In each case it is necessary to heat to at least 100° C. and the enantiomeric excesses are insufficient for industrial application:
Reetz et al. on the one hand (Reetz M. T.; Moulin D.; Gosberg A. Org. Lett. 2001, 3, 4083), Frost et al. on the other hand (Chapman C. J.; Hargrave J. D.; Bish G.; Frost C. G. Tetrahedron, 2008, 64, 9528; Chapman C. J.; Wadsworth K. J.; Frost C. G. J. Organomet. Chem. 2003, 680, 206) use dioxane in the presence of water performing the role of proton donor, and a very weak base, NaF.
Van der Eycken et al. (Noël T.; Gök Y.; Van der Eycken, J. Tetrahedron Asymmetry 2010, 21, 540) use the same conditions as Reetz and Frost apart from the ligand and do not obtain better results.
Darses et al. (Navarre L.; Darses S.; Genêt, J. P. Angew. Chem. Int. Ed 2004, 43, 719 and Navarre L.; Martinez R.; Genet J. P.; Darses S. J. Am. Chem. Soc. 2008, 130, 6159) use toluene in the presence of guaiacol as proton source, without NaF.

None of these preparation methods allows enantiomeric excesses above 95% to be obtained.

Sibi et al. obtain β-amino acids by the addition of arylboronic acids, catalysed by a rhodium/(S)-difluorphos combination, in dioxane, heating to a temperature of 50° C., with phthalimide as the proton donor (Sibi M. P.; Tatamidani H.; Patil K. Org. Lett. 2005, 7, 2571). Once again, heating is necessary and the enantiomeric excesses do not exceed 91%.
It therefore appears that in all the cases cited above, the reaction mixture must be heated. Moreover, the enantiomeric excesses are not sufficient to give rise to an industrial process for the synthesis of chiral amino acids.
The purpose of the invention is to supply a process for preparing chiral α-amino acids and β-amino acids with enantiomeric excesses above 95%.
The purpose of the invention is to obtain good chemical yields in the preparation of these chiral amino acids.
The purpose of the invention is to carry out the synthesis under mild temperature conditions.

According to a general aspect, the invention relates to the use of a solvent and a proton donor element, the pKa of which in water is above 7, in the presence of a base belonging to an acid/base pair the pKa of which pKa in water is above 4, for carrying out a process for preparing chiral compounds consisting of α- or β-amino acids or derivatives thereof with an enantiomeric excess of at least 95%, by reacting a starting product consisting of an α-aminoacrylate with an organoboron derivative, with the aid of an electron-poor biphosphorus ligand in the presence of a catalyst containing a transition metal, at a temperature in the range from −20° C. to 70° C.

The invention is based on the unexpected finding that the use of a base makes it possible to obtain very good enantiomeric excesses. In this synthesis, an α-aminoacrylate and an organoboron compound are reacted in a basic medium. This reaction is catalysed by a complex containing a transition metal and a biphosphorus ligand. The chirality is carried by the ligand. The solvent makes it possible to dissolve all the species involved. A proton donor element is also employed. The products obtained are chiral α- or β-amino acids, obtained in the form of species in which the amine and carboxylic acid functions are protected.
In the process of the invention,
All the chemical species involved in the reaction are initially mixed and dissolved in the solvent; it is therefore a “one-pot” process.
According to the examples carried out, the reaction mixture is not heated or is heated to a fairly mild temperature.
The chiral amino acids are obtained with good chemical yields.
The chiral amino acids are obtained with excellent enantiomeric excesses.
   This therefore means that industrial application can be envisaged.

The expression “proton donor element” denotes an acid entity in the Brønsted sense. The proton donor element is therefore a chemical species that is able to give up a proton to another species in the reaction mixture.

The term “solvent” denotes a liquid capable of dissolving one or more chemical species and capable of keeping the temperature of the reaction mixture uniform at every point during the chemical conversion.

The expression “chiral compounds consisting of α- or β-amino acids or derivatives thereof” denotes the α- or β-amino acids, unsubstituted, or substituted with one or more elements or groups, which may be identical or different, said substituted or unsubstituted α- or β-amino acids being chiral, said groups not themselves consisting of α- or β-amino acids.

The expression “enantiomeric excess” denotes the physical quantity quantifying the optical purity of a compound obtained during a chemical reaction.

The quantity of the dominant enantiomer and the quantity of the opposing enantiomer are measured. The definition of the enantiomeric excess, designated “ee”, is given by the following expression:

ee=|η_{R}−η_{S}|×100% where η_{R} and η_{S} denote mole fractions of the enantiomers (R) and (S) such that η_{R}+η_{S}=1.

The expression “α-aminoacrylates” includes the protected α-amino acids and β-amino acids, which can be represented by the following formula

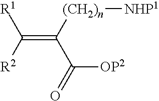

in which R¹, R², P¹, P² have the meanings stated below. When n is equal to 0 it is an α-amino ester, and when n is equal to 1 it is a β-amino ester.

The expression “organoboron compound” denotes a chemical compound having a boron-carbon bond. Several families of organoboron compounds are used.

The organoboron compounds used in the present invention are either commercial compounds, or compounds accessible by synthesis. They are compounds that are stable and easily handled.

The term “ligand” denotes a molecule bearing chemical functions permitting it to bind to a metal atom or to a central metal cation.

The expression “biphosphorus ligand” denotes a ligand bearing two phosphorus atoms. These ligands belong to the diphosphine family. Each phosphorus atom is therefore trivalent and has a non-binding electron doublet that can be given up to the transition metal.

The ligands used in the invention are diphosphines that are chiral by atropisomerism; they therefore have axial chirality. The enantiomerism is connected with the prevention of rotation about a single bond. Steric hindrance of the substituents around this bond is such that at normal temperature the rate of interconversion is low enough for the two enantiomers to be separated. Owing to their conformational flexibility, these diphosphines can easily be complexed with a large number of transition metals for numerous enantioselective catalytic reactions. The atropisomeric systems therefore prove to be of great importance in asymmetric synthesis.
The biphosphorus ligand is therefore a bidentate ligand (denticity equal to two). In association with a metal atom or a metal cation, the biphosphorus ligand forms a coordination complex.

The expression “electron-poor biphosphorus ligand” denotes a ligand which is a diphosphine, one substituent of which bears electron-attracting groups.

The electron-poor character of the biphosphorus ligand is quantified using the scale defined by the phosphorus/selenium coupling constant designated J_{P—Se} (D. W. Allen, B. F. Taylor, J. Chem. Soc., Dalton Trans. 1982, 51-54). The ligand is selected such that J_{P—Se}>720 Hz.
The biphosphorus ligand is also qualified as an electron-poor ligand by correlation with the scale of wavenumbers corresponding to the vibration frequency ν(C═O) of the rhodium complexes of structure [RhCl(diphosphine)(CO)]. The ligand is selected in such a way that the vibration frequency ν(C═O) is above 2010 cm⁻¹ (S. Vastag, B. Heil, L. Markó, J. Mol. Catal. 1979, 5, 189-195).

The term “catalyst” denotes a complex containing a transition metal. This complex is capable of increasing the rate of the reaction. The transition metal is selected from groups 8, 9 and 10 of the periodic table.

The synthesis of α- and β-amino acids is a “one-pot” synthesis. No reaction intermediate is isolated. However, this synthesis comprises two key steps.

The first key step corresponds to the 1,4-addition of the organoboron compound on α-aminoacrylate. During this step, the carbon-containing group attached to the boron atom is transferred to the α-aminoacrylate. A new carbon-carbon bond is therefore formed between this group, supplied by the organoboron compound, and the α-aminoacrylate. A reaction intermediate is obtained, probably of the oxa-π-allylmetal type. This complex is chiral since the metal/chiral ligand association is involved.

During the second key step, the proton donor element gives up its proton to the chiral reaction intermediate previously formed. This protonation is diastereoselective: this is what controls the chiral centre.

According to another advantageous embodiment of the invention, the proton donor is the solvent and is in particular selected from primary, secondary or tertiary alcohols containing from 1 to 8 carbon atoms and in particular selected from methanol, ethanol, n-propanol, n-butanol, isopropanol, sec-butanol, isobutanol and tert-butanol.

According to a particular embodiment, the proton donor element defined above is the solvent itself It is selected from the three classes of alcohols.

Generally, alcohols are good solvents. In the invention, they make it possible to dissolve some or all of the compounds involved in the procedure.
By the general term “alcohol” is meant primary, secondary, tertiary, and in particular secondary, alcohols.

If the alcohol belongs to the class of primary alcohols, it has from 1 to 8 carbon atoms and is in particular selected from methanol, ethanol, n-propanol and n-butanol. The reaction products are then generally obtained with enantiomeric excesses above 95%, this statement being non-limitative.

If the alcohol belongs to the class of secondary alcohols, it has from 3 to 8 carbon atoms and is selected from isopropanol or sec-butanol, and in particular isopropanol. The reaction products are then advantageously obtained with enantiomeric excesses of the order of 99%, this statement being non-limitative.

If the alcohol belongs to the class of tertiary alcohols, it has from 4 to 8 carbon atoms. The enantiomeric excesses are of the order of 98%, this statement being non-limitative.

According to another embodiment of the invention, the base is selected from: MHCO₃, M₂CO₃, MOAc, MOH, M′CO₃, R^{c}R^{d}R^{e}N,
M denoting a single-charge cation belonging to the alkali family and selected from the lithium ion Li⁺, sodium ion Na⁺, potassium ion K⁺, caesium ion Cs⁺,
M′ denoting a double-charge cation belonging to the alkaline-earth family and selected from the calcium ion Ca²⁺ and the barium ion Ba²⁺,
R^{c}, R^{d}, R^{e} being selected from H or a carbon chain in particular having 1 to 6 carbon atoms, selected independently of one another.

When the reaction is carried out without a base, only traces of product are observed.

According to another embodiment of the invention, the transition metal is selected from rhodium, iridium or palladium.

Advantageously, the catalytic complexes used in the invention and containing the element rhodium are selected from [RhCl(C₂H₄)₂]₂, [RhCl(cod)]₂ where cod denotes 1,5-cyclooctadiene, [RhCl(nbd)]₂ where nbd denotes norbornadiene, [RhCl(coe)₂]₂ where coe denotes cyclooctene, [RhCl(CO)₂]₂, [RhOH(cod)]₂, [RhOH(nbd)]₂, [Rh(acac)(C₂H₄)₂]₂ where acac denotes acetylacetonate, [Rh(acac)(coe)₂], [Rh(acac)(cod)], [Rh(cod)₂]BF₄, [Rh(nbd)₂]BF₄, [Rh(cod)₂]PF₆, [Rh(cod)₂]ClO₄, [Rh(cod)₂]OTf where TfO denotes trifluoromethanesulphonate, [Rh(cod)₂]BPh₄, in particular the dimer chlorobis(ethylene)rhodium(I).

According to another embodiment of the invention, the catalyst containing a transition metal comprises [RhCl(C₂H₄)₂]₂.
The complex containing the transition metal forms an association with the electron-poor biphosphorus ligand. This association catalyses the chemical conversion and permits control of the absolute configuration of the asymmetric carbon generated.

According to a particular embodiment of the invention, the biphosphorus ligand is selected from: (R)-Binap, (S)-Binap, (R)-Difluorphos, (S)-Difluorphos, (R)-Synphos, (S)-Synphos, (R)-MeO-biphep, (S)-MeO-biphep, (R)-Segphos, (S)-Segphos.

These are diphosphines that are chiral by atropisomerism; they therefore have axial chirality.
The (R)- and (S)-2,2′-bis(diphenylphosphino)-1,1′-binaphthyl, called (R)- and (S)-BINAP, are in particular described in “Miyashita, A.; Yasuda, A.; Souchi, T.; Ito, T.; Noyori, R. J. Am. Chem. Soc. 1980, 102, 7932”. The coupling constant J_{P—Se} is equal to 738 Hz. The (R)- and (S)-BINAP are represented below:

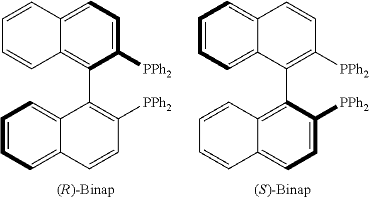

The (R)- and (S)-Difluorphos™ are described in “S. Jeulin, S. Duprat de Paule, V. Ratovelomanana-Vidal, J.-P. Genêt, N. Champion, P. Dellis, Angew. Chem. Int. Ed. 2004, 43, 320-325” and “S. Duprat de Paule, N. Champion, V. Vidal, J.-P. Genet, P. Dellis, Patent WO03029259, 2003”. The coupling constant J_{P—Se} is equal to 749 Hz. They have the formulae:

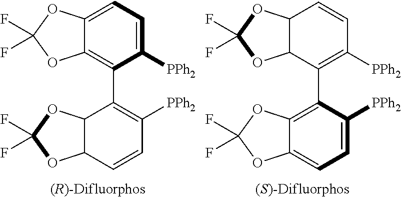

The (R)- and (S)-Synphos™ are described in “S. Jeulin, S. Duprat de Paule, V. Ratovelomanana-Vidal, J.-P. Genêt, N. Champion, P. Dellis, Angew. Chem. Int. Ed. 2004, 43, 320-325” and “S. Duprat de Paule, N. Champion, V. Vidal, J.-P. Genet, P. Dellis, Patent WO03029259, 2003”. The coupling constant J_{P—Se} is equal to 740 Hz. They have the formulae:

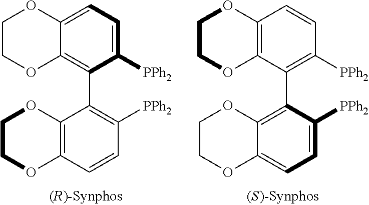

The (R)- and (S)-MeO-biphep are described in “R. Schmid, J. Foricher, M. Cereghetti, P. Schönholzer, Helv. Chim. Acta 1991, 74, 370-389”. The coupling constant J_{P—Se} is equal to 742 Hz. They have the formulae:

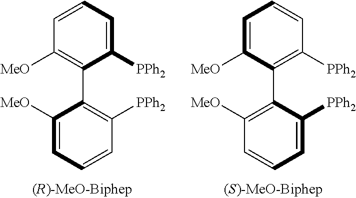

The (R)- and (S)-Segphos are described in “T. Saito, T. Yokozawa, T. Ishizaki, T. Moroi, N. Sayo, T. Miura, H. Kumobayashi, Adv. Synth. Catal. 2001, 343, 264-267”. The coupling constant J_{P—Se} is equal to 738 Hz. They have the formulae:

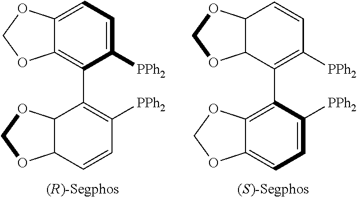

During the synthesis, the organoboron compound makes it possible to create a carbon-carbon bond in position β relative to the carbon of the carboxyl function, on the dehydroalanine derivative.

According to an advantageous embodiment of the invention, the organoboron derivative has the following formula:

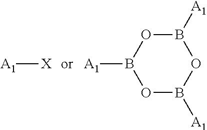

in which:
A₁ is selected from:
1. the aryls having rings with 6 to 15 carbon atoms, optionally substituted:
   with one or more halogen atoms comprising fluorine, chlorine, bromine or iodine,
   with hydroxy, amino or thio radicals optionally protected by “ad hoc” protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals with 1 to 15 carbon atoms,
   with optionally substituted alkenyl radicals with 1 to 15 carbon atoms,
   with optionally substituted alkynyl radicals with 1 to 15 carbon atoms,
   with optionally substituted aryls with 6 to 12 carbon atoms,
   with optionally substituted aromatic or non-aromatic heterocycles with 2 to 12 carbon atoms,
in which R^{a} and R^{b}, which may be identical or different, represent linear or branched, aromatic or heterocyclic non-aromatic or aromatic alkyl, alkenyl, alkynyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted linear or branched alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted aryls with 6 to 12 carbon atoms,
   with optionally substituted aromatic or non-aromatic heterocycles having from 2 to 12 carbon atoms.
2. A₁ is further selected from the heterocycles or the heteroaryls having rings with 2 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
3. A₁ is further selected from linear or branched alkenyls having from 1 to 12 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms.
4. A₁ is further selected from linear or branched alkynyls having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
5. A₁ is further selected from linear or branched alkyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
   X is selected from B(OH)₂, B(OR)₂, BF₃M, B(OR′)₃M,
      in which:
   R is an alkyl group having from 1 to 14 carbon atoms, (OR)₂ optionally forming a ring between the two oxygen atoms. The groups (OR)₂ in particular originate from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and esters thereof,
      or (OR)₂ in particular originates from diacids such as 2,2′-(methylazanediyl)diacetic acid (mida),
   R′ is an alkyl group having from 1 to 14 carbon atoms, (OR)₃ optionally forming a ring between two of the oxygen atoms or a dicyclic ring between the three oxygen atoms. The groups (OR)₃ in particular originate from triols such as 2-(hydroxymethyl)-2-methylpropane-1,3-diol,
   M represents the lithium ion Li⁺, sodium ion Na⁺, potassium ion K⁺, caesium ion Cs⁺, ammonium ion R^{c}R^{d}R^{e}R^{f}N⁺ where R^{c}, R^{d}, R^{e}, R^{f} are selected from H or a saturated carbon chain in particular having 1 to 6 carbon atoms selected independently of one another,
      and in particular A₁-X represents A₁-B(OH)₂, A₁-B(OR)₂ or A₁-BF₃K,
      in which A₁ has the same meaning as above.
      In the formula A₁-X:
   either the boron atom is trivalent and the term X denotes B(OH)₂. A₁-X is then a boronic acid (compound I-A) having the formula

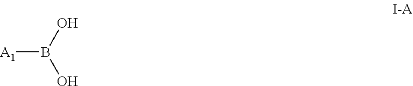

or the boron atom is trivalent and the term X denotes B(OR)₂. A₁-X is then a boronic ester (compound I-B) having the formula, with R having the same meaning as above,

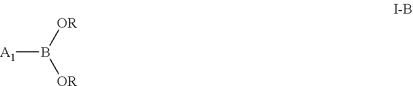

or the boron atom is tetravalent and the term X denotes BF₃M. A₁-X is then a trifluoroborate (compounds I-C) having the formula, with M having the same meaning as above,

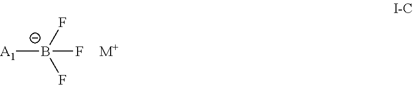

or the boron atom is tetravalent and the term X denotes B(OH)₃M. A₁-X is then a trihydroxyborate (compounds I-D) having the formula, with M having the same meaning as above,

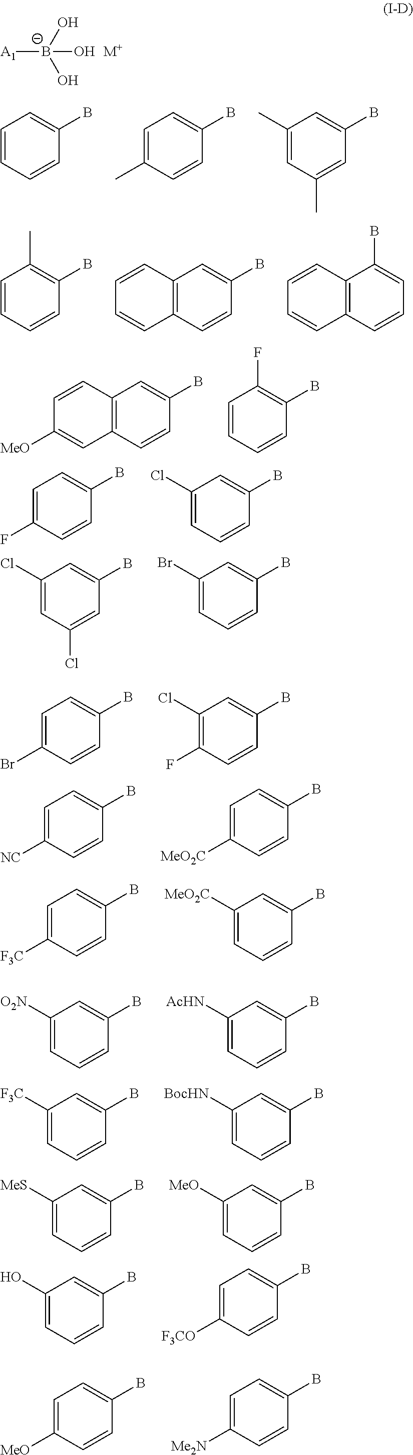

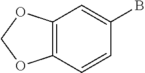

Examples of “A₁-B” units are shown below:

According to a particular embodiment of the invention, synthesis of the chiral amino acids leads to a yield equal to at least 40% and in particular above 70%.

According to a particular embodiment, the starting product consists of an α-aminoacrylate.

According to another particular embodiment, the starting product consists of an α-aminomethyl acrylate.

According to a particular embodiment of the invention, the starting product is a compound of formula:

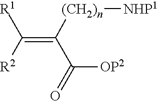

in which:
R¹ and R² are selected, independently of one another, from alkyl or aromatic groups containing from 1 to 10 carbon atoms, provided that at least R¹ or R² is a hydrogen,
n is equal to 0 or 1,
P¹ is an amine protective group selected from:
   COR³, in which R³ represents a linear or branched alkyl, alkenyl, alkynyl group; benzyl, phthalimido (in this case NH is replaced with N) optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
   in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
   COOR⁴, in which R⁴ represents an alkyl group, more particularly methyl, ethyl, propyl, benzyl, tert-butyl, but also alkenyl, alkynyl, linear or branched, benzyl optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
   in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is a carboxylic acid protective group in particular selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
P² is further selected from linear or branched alkenyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
P² is further selected from linear or branched alkynyl groups having from 1 to 15 carbon atoms and optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
P² is further selected from linear or branched benzyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
P² is further selected from linear or branched silyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
   with one or more halogen atoms comprising fluorine, chlorine, bromine or iodine,
   with hydroxy, amino or thio radicals optionally protected by ad hoc protective groups,
   with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
   with optionally substituted alkyl radicals having from 1 to 15 carbon atoms,
   with optionally substituted linear or branched alkenyl radicals having from 1 to 15 carbon atoms,
   with optionally substituted linear or branched alkynyl radicals having from 1 to 15 carbon atoms,
   with optionally substituted linear or branched aryl radicals having from 6 to 12 carbon atoms,
   with optionally substituted aromatic or non-aromatic heterocycles having from 2 to 12 carbon atoms.
P¹ is in particular selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine.
   P² is in particular selected from linear or branched alkyl groups having from 1 to 15 carbon atoms, optionally substituted, and in particular methyl, ethyl, isopropyl, tert-butyl and benzyl.
   The amine and carboxylic acid functions are protected. The aim is to avoid undesirable side reactions that would modify these two functions and would consequently decrease the chemical yields. Deprotection can be carried out subsequently, once the desired product is obtained.

The term “P¹” denotes a protective group of the amine function.

If P¹ represents a —COR³ group, then the amine function is protected in the form of amide.

If P¹ represents a —COOR⁴ group, then the amine function is protected in the form of carbamate.

The term “P²” denotes a protective group of the carboxylic acid function, which is protected in the form of ester corresponding to the “—COOP²” group.

The subscript designated “n” corresponds to the number of methylene groups between the protected amine function and the protected acid function. “n” is either zero, or is equal to one.
If n=0, the starting compound is an α-aminoacrylate, which leads to a chiral α-amino ester being obtained,
If n=1, the starting compound is an α-aminomethyl acrylate, which leads to a chiral β-amino ester being obtained.

According to another embodiment of the invention, the chiral α- and β-amino acids or derivatives thereof have the formula:

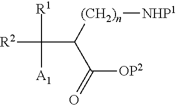

in which A₁, P¹, P² have the same meanings as stated above,
n is equal to 0 or 1,
R¹ and R² are selected, independently of one another, from alkyl or aromatic groups containing from 1 to 10 carbon atoms, provided that at least R¹ or R² is a hydrogen.

According to a particular embodiment of the invention, the starting product has the formula:

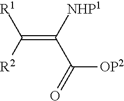

in which:
P¹, P² have the same meanings as stated above,
R¹ and R² are selected, independently of one another, from alkyl or aromatic groups containing from 1 to 10 carbon atoms, provided that at least R¹ or R² is a hydrogen,
   and is used for preparing a chiral derivative of formula:

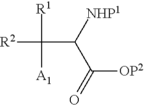

in which A₁, P¹, P², R¹ and R² have the same meanings as stated above.

If R¹ and R² are identical, a chiral centre is obtained at α of the carbonyl. The synthesis therefore produces a mixture of enantiomers with a very large excess of one relative to the other.

If R¹ and R² are different, a second chiral centre is induced. Two chiral centres are then obtained, at α and at β of the carbonyl. In this case, a mixture of diastereoisomers is produced.

The equation of the reaction is shown diagrammatically below, where the term “L*” represents the chiral biphosphorus ligand:

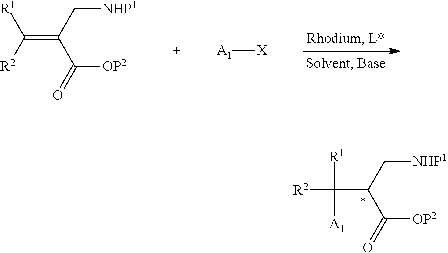

According to another embodiment of the invention, the starting product has the formula:

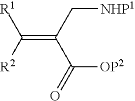

in which:
P¹, P² have the same meanings as stated above,
R¹ and R² are selected, independently of one another, from alkyl or aromatic groups containing from 1 to 10 carbon atoms, provided that at least R¹ or R² is a hydrogen,
   and is used for preparing a chiral derivative of formula:

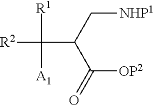

in which A₁, P¹, P², R¹ and R² have the same meanings as stated above.

If R¹ and R² are identical, a chiral centre is obtained at α of the carbonyl. The synthesis therefore produces a mixture of enantiomers with a very large excess of one relative to the other.

If R¹ and R² are different, a second chiral centre is induced. Two chiral centres are then obtained, at α and at β of the carbonyl. In this case, a mixture of diastereoisomers is produced.

The equation of the reaction is shown diagrammatically below, where the term “L*” represents the chiral biphosphorus ligand:

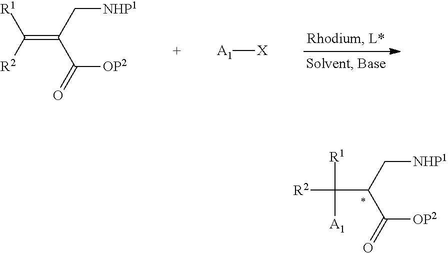

According to an advantageous embodiment of the invention, P¹ is an amine protective group as defined above and in particular selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine.

For example, the tert-butyloxycarbonyl group is selected as the protective group of the amine function. The starting products are represented by the following formula:

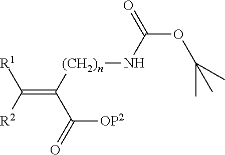

P², R¹ and R² have the same meanings as stated above.

According to an advantageous embodiment of the invention, P², the protective group of the carboxylic acid function, is as defined above and is in particular selected from linear or branched alkyl groups having from 1 to 15 carbon atoms, optionally substituted, and in particular methyl, ethyl, isopropyl, tert-butyl and benzyl.

For example, the isopropyl group is selected as the protective group of the acid function, and the formula of the starting product is represented as follows:

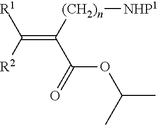

P¹, R¹ and R² have the same meanings as stated above.

According to a particular embodiment of the invention, R¹ and R² are hydrogen atoms.
If the starting product is an α-aminoacrylate, an α-amino ester is obtained.
If the starting product is an α-aminomethyl acrylate, a β-amino ester is obtained.

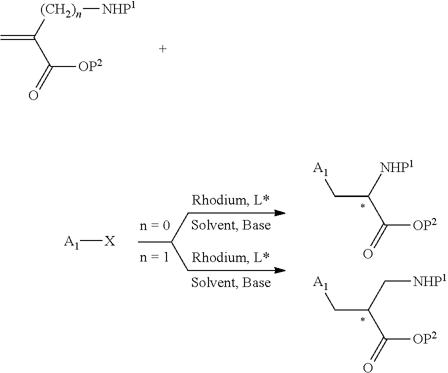

In the above diagram, the term “L*” represents the chiral biphosphorus ligand.

According to another embodiment, the synthesis is carried out at a temperature in the range from −20° C. to 70° C.
The synthesis is advantageously carried out at a temperature in the range from −20° C. to 40° C., when the organoboron derivative is a boronic acid I-A, and in particular from 0 to 40° C.
The synthesis is advantageously carried out at a temperature in the range from 40° C. to 60° C., when the organoboron derivative is a boronic ester I-B.
The synthesis is advantageously carried out at a temperature in the range from 40° C. to 70° C., when the organoboron derivative is a borate I-C or I-D.
   The formulae I-A, I-B, I-C and I-D are as defined above.

According to an advantageous embodiment of the invention, the enantiomeric excesses obtained are above 98.5%.

According to one of its aspects, the invention relates to a process for preparing chiral α- or β-amino acids or derivatives thereof with an enantiomeric excess of at least 95%, comprising a step of reacting a starting product consisting of an α-aminoacrylate or of an α-aminomethyl acrylate with an organoboron derivative, with the aid of a solvent and a proton donor element, the pKa of which in water is above 7, in the presence of a base belonging to an acid/base pair with pKa in water above 4, an electron-poor biphosphorus ligand, and a catalyst containing a transition metal, at a temperature in the range from −20° C. to 70° C., allowing protected chiral α- or β-amino acids or derivatives thereof to be obtained, and an optional deprotection step of the protected chiral amino acids obtained or derivatives thereof.

The purpose of the present invention is a process for preparing chiral α- or β-amino acids or derivatives thereof comprising a step of reacting a starting product consisting of a derivative of α-aminoacrylate with an organoboron derivative optionally followed by a deprotection step.
In this process, a carbon-carbon bond is created by the addition of the carbon-containing group supplied by the organoboron compound onto the α-aminoacrylate or α-aminomethyl acrylate. This addition is followed by protonation brought about by the proton donor element.
It is a “one-pot” procedure. The following equation allows the chemical conversion to be modelled. In this equation, A₁-X, R¹, R², P¹, P² are as defined above. In the following diagram, the term “L*” represents the chiral biphosphorus ligand.

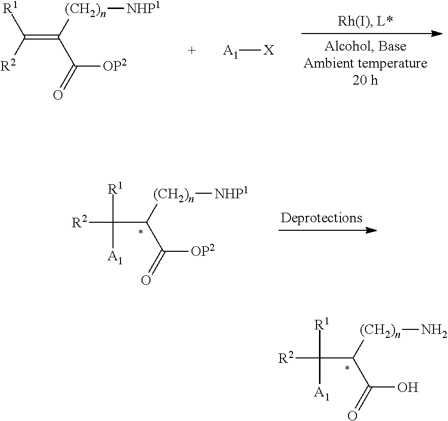

According to another embodiment, in the process of the invention, the proton donor is the solvent, in particular selected from primary, secondary or tertiary alcohols with 1 to 8 carbon atoms and in particular selected from methanol, ethanol, n-propanol, n-butanol, isopropanol, sec-butanol, isobutanol and tert-butanol.

According to an advantageous embodiment of the invention, a polar protic solvent, which is an alcohol, will be used. Owing to its polar protic character, the alcohol can constitute not only the solvent, capable of dissolving some or all of the species used in the reaction, but also the proton donor element defined above.

According to another embodiment of the process of the invention, the base is selected from: MHCO₃, M₂CO₃, M′CO₃, MOH, MOAc, R^{c}R^{d}R^{e}N.
M denoting a single-charge cation belonging to the alkali family and selected from the lithium ion Li⁺, sodium ion Na⁺, potassium ion K⁺, caesium ion Cs⁺,
M′ denoting a double-charge cation belonging to the alkaline-earth family and selected from the calcium ion Ca²⁺ and the barium ion Ba²⁺,
R^{c}, R^{d}, R^{e} being selected from H or a carbon chain in particular having 1 to 6 carbon atoms, selected independently of one another.
   The base is selected from carbonates, hydroxides, amines, acetates, hydrogen carbonates, and is in particular sodium hydrogen carbonate.

The present invention relates to the use of a catalyst derived from a complex containing a transition metal, in particular selected from rhodium, iridium or palladium, and in particular rhodium.

The catalyst containing a transition metal comprises the complexes containing the element rhodium. It is selected, for example, from [RhCl(C₂H₄)₂]₂, [RhCl(cod)]₂ where cod denotes 1,5-cyclooctadiene, [RhCl(nbd)]₂ where nbd denotes norbornadiene, [RhCl(coe)₂]₂ where coe denotes cyclooctene, [RhCl(CO)₂]₂, [RhOH(cod)]₂, [RhOH(nbd)]₂, [Rh(acac)(C₂H₄)₂]₂ where acac denotes acetylacetonate, [Rh(acac)(coe)₂], [Rh(acac)(cod)], [Rh(cod)₂]BF₄, [Rh(nbd)₂]BF₄, [Rh(cod)₂]PF₆, [Rh(cod)₂]ClO₄, [Rh(cod)₂]OTf where TfO denotes trifluoromethanesulphonate, [Rh(cod)₂]BPh₄.

According to another embodiment of the process of the invention, the catalyst containing a transition metal comprises [RhCl(C₂H₄)₂]₂.

According to an advantageous embodiment of the protocol of the invention, the biphosphorus ligand is selected from: (R)-Binap, (S)-Binap, (R)-Difluorphos, (S)-Difluorphos, (R)-Synphos, (S)-Synphos, (R)-MeO-biphep, (S)-MeO-biphep, (R)-Segphos, (S)-Segphos and in particular (S)- or (R)-Difluorphos.

The scales for quantifying the electron-poor character of the ligand are as defined above.

According to an advantageous embodiment of the protocol of the invention, the organoboron derivative has the following formula:

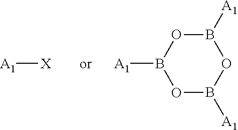

in which:
A₁ is as defined above,
X is selected from B(OH)₂, B(OR)₂, BF₃M, B(OR′)₃M,
   in which:
R is an alkyl group having from 1 to 14 carbon atoms, (OR)₂ optionally forming a ring between the two oxygen atoms. The groups (OR)₂ in particular originate from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and esters thereof,
   or (OR)₂ in particular originates from diacids such as 2,2′-(methylazanediyl)diacetic acid (mida),
R′ is an alkyl group having from 1 to 14 carbon atoms, (OR)₃ optionally forming a ring between two of the oxygen atoms or a dicyclic ring between the three oxygen atoms. The groups (OR)₃ in particular originate from triols such as 2-(hydroxymethyl)-2-methylpropane-1,3-diol,
M represents the lithium ion Li⁺, sodium ion Na⁺, potassium ion K⁺, caesium ion Cs⁺, ammonium ion R^{c}R^{d}R^{e}R^{f}N⁺ where R^{c}, R^{d}, R^{e}, R^{f} are selected from H or a saturated carbon chain in particular having 1 to 6 carbon atoms selected independently of one another,
   and in particular A₁-B(OH)₂, A₁-B(OR)₂ or A₁-BF₃K,
   in which A₁ has the same meaning as above.
   In the formula A₁-X,
either the boron atom is trivalent and the term X denotes B(OH)₂. A₁-X is then a boronic acid (compound I-A) having the formula:

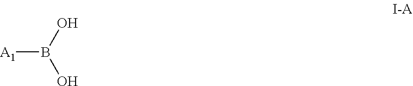

or the boron atom is trivalent and the term X denotes B(OR)₂. A₁-X is then a boronic ester (compound I-B) having the formula, with R having the same meaning as above,

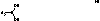

or the boron atom is tetravalent and the term X denotes BF₃M. A₁-X is then a trifluoroborate (compounds I-C) having the formula, with M having the same meaning as above,

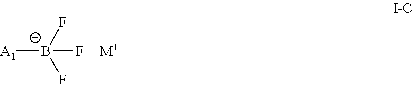

or the boron atom is tetravalent and the term X denotes B(OH)₃M. A₁-X is then a trihydroxyborate (compounds I-D) having the formula, with M having the same meaning as above,

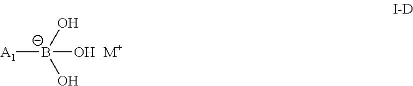

Examples of “A₁-B” units are those stated above.

The process of the present invention gave yields equal to at least 40%, and in particular yields above 70%.

According to one embodiment of the process of the invention, the starting product is an α-aminoacrylate.

According to another embodiment of the process of the invention, the starting product is an α-aminomethyl acrylate.

The invention relates to a process in which the starting product is a compound of formula:

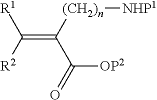

in which:
P¹ and P² are as defined above,
n is equal to 0 or 1,
R¹ and R² are selected, independently of one another, from alkyl or aromatic groups containing from 1 to 10 carbon atoms, provided that at least R¹ or R² is a hydrogen.

The invention relates in particular to a process in which the chiral α- or β-amino acids or derivatives thereof have the formula:

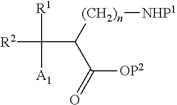

in which:
A₁, P¹ and P² have the same meanings as defined above,
n is equal to 0 or 1,
R¹ and R² are selected, independently of one another, from alkyl or aromatic groups containing from 1 to 10 carbon atoms, provided that at least R¹ or R² is a hydrogen.

The invention relates in particular to a process in which the starting product has the formula:

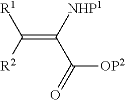

in which:
P¹ and P² are as defined above,
R¹ and R² are selected, independently of one another, from alkyl or aromatic groups containing from 1 to 10 carbon atoms, provided that at least R¹ or R² is a hydrogen,
   and is used for preparing a chiral derivative of formula:

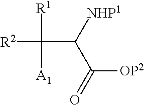

in which A₁, P¹, P², R¹ and R² have the same meanings as defined above.
The equation of the reaction is shown diagrammatically below, where the term “L*” represents the chiral biphosphorus ligand:

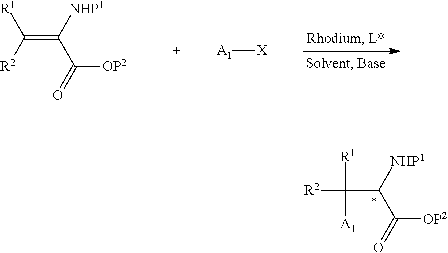

According to another embodiment of the process of the invention, the starting product has the formula:

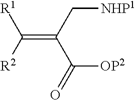

in which:
P¹ and P² are as defined above,
R¹ and R² are selected, independently of one another, from alkyl or aromatic groups containing from 1 to 10 carbon atoms, provided that at least R¹ or R² is a hydrogen,
   and is used for preparing a chiral derivative of formula:

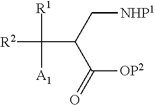

in which A₁, P¹, P², R¹ and R² have the same meanings as defined above.
The equation of the reaction is shown diagrammatically below, where the term “L*” represents the chiral biphosphorus ligand:

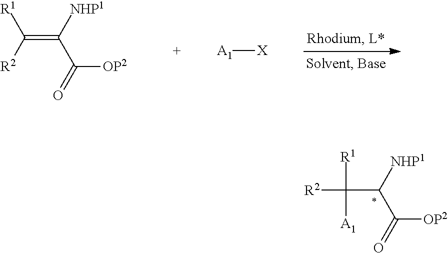

The invention relates to a process in which P¹ is a protective group of the amine function. P¹ is as defined above. It is in particular selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine.

In the process of the invention, P² is a protective group of the carboxylic acid function. P² is as defined above. It is in particular selected from the methyl, ethyl, propyl, isopropyl, butyl, isobutyl, sec-butyl, tert-butyl, and in particular, isopropyl groups.

According to a particular embodiment of the process of the invention, R¹ and R² are hydrogen atoms.

According to a particular embodiment of the process of the invention, the synthesis is carried out at a temperature in the range from −20° C. to 70° C.

The process is carried out at a temperature in the range from −20° C. to 70° C.
The synthesis is in particular carried out at a temperature in the range from −20° C. to 40° C., when the organoboron derivative is a boronic acid I-A, and in particular from 0 to 40° C.
The synthesis is in particular carried out at a temperature in the range from 40° C. to 60° C., when the organoboron derivative is a boronic ester I-B.
The synthesis is in particular carried out at a temperature in the range from 40° C. to 70° C., when the organoboron derivative is a borate I-C or I-D.
   The formulae I-A, I-B, I-C and I-D are as defined above.

The process makes it possible to obtain enantiomeric excesses above 98.5%.

The invention relates in particular to a process for preparing a compound of formula:

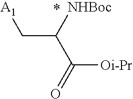

in which A₁ is as defined above,
by reaction between a compound of formula:

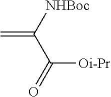

and a boronic acid of formula:

A₁-B(OH)₂

in which A₁ is as defined above,
in a medium comprising:

a protic solvent selected from primary, secondary or tertiary alcohols containing from 1 to 8 carbon atoms and in particular selected from methanol, ethanol, n-propanol, n-butanol, isopropanol, sec-butanol, isobutanol and tert-butanol,

a base selected from: MHCO₃, M₂CO₃, MOAc, MOH, M′CO₃, R^{c}R^{d}R^{e}N,
M denoting a single-charge cation belonging to the alkali family and selected from the lithium ion Li⁺, sodium ion Na⁺, potassium ion K⁺, caesium ion Cs⁺,
M′ denoting a double-charge cation belonging to the alkaline-earth family and selected from the calcium ion Ca²⁺ and the barium ion Ba²⁺,
R^{c}, R^{d}, R^{e} being selected from H or a carbon chain in particular having 1 to 6 carbon atoms, selected independently of one another,

[RhCl(C₂H₄)₂]₂, and

a biphosphorus ligand selected from: (R)-Binap, (S)-Binap, (R)-Difluorphos, (S)-Difluorphos, (R)-Synphos, (S)-Synphos, (R)-MeO-biphep, (S)-MeO-biphep, (R)-Segphos, (S)-Segphos.

The temperature used is in the range 20° C. to 40° C. and the reaction time is from 30 min to 2 days.
The yield is above 40% and the enantiomeric excess is above 98.5%.
The equation representing the chemical conversion of the preferred embodiment is shown below, where the term “L*” represents the chiral biphosphorus ligand:

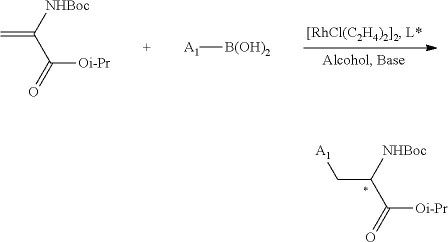

The invention relates in particular to a process for preparing a compound of formula:

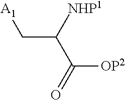

in which

A₁ is a group of formula:

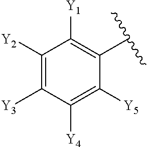

in which Y₁, Y₂, Y₃, Y₄ and Y₅ are selected independently of one another from:
a hydrogen,
an alkyl or aromatic group comprising 1 to 10 carbon atoms,
a halogen,
—CN,
—CO₂Me,
—CF₃,
—COMe,
—NO₂,
—NHAc,
—NHBoc,
—SMe,
—OMe,
—OH,
—OCF₃, and
—NMe₂

In this embodiment, A₁ can be derived from the naphthyl group or can be heteroaromatic, and is in particular selected from the groups shown below:

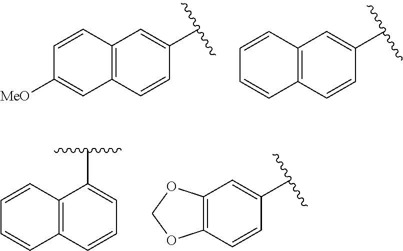

P¹ is an amine protective group as defined above, and in particular P¹ is selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine,
P² is selected from methyl, ethyl, n-propyl, isopropyl, butyl, tert-butyl, benzyl and in particular isopropyl,
   by reaction between a compound of formula:

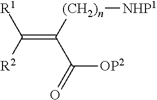

in which R¹=R²=H,
n=0,
in which P¹ and P² have the same meanings as stated above,
and a boronic acid, of formula: A₁-B(OH)₂
in which A₁ is as defined above,
in a medium comprising:

a protic solvent selected from methanol, ethanol, n-propanol, n-butanol, isopropanol, sec-butanol, isobutanol and tert-butanol,

NaHCO₃,

[RhCl(C₂H₄)₂]₂, and

the biphosphorus ligand (S)- or (R)-Difluorphos.

The reaction mixture is maintained at a temperature in the range from 20° C. to 30° C., for a time in the range from 30 minutes to 25 hours.
These compounds are obtained with a yield equal to at least 40%, and in particular above 70%.
The enantiomeric excess is above 98.5%.
The equation representing the chemical conversion according to these examples is shown below.

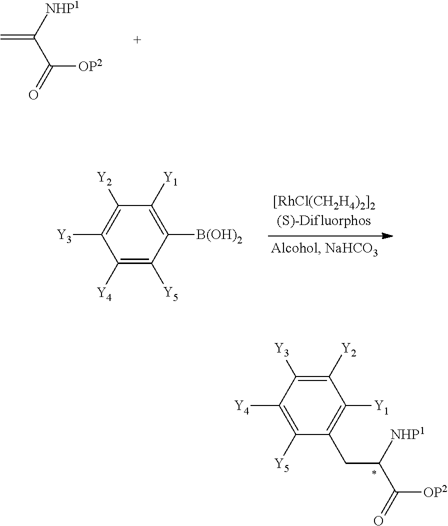

The invention relates in particular to a process for preparation in which the organoboron compound is phenylboronic acid, the alcohol is isopranol, the amine protective group is Boc and the protective group of the acid is isopropyl.
The equation of this example of reaction is shown below:

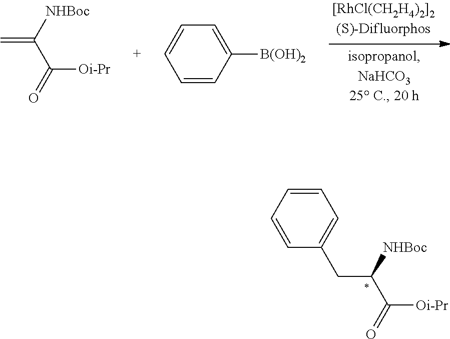

### EXPERIMENTAL SECTION

### Preparation of the Compounds

The absolute configuration is determined by comparison with data in the literature for the sign of the rotatory power when the compounds are known and described. Thus, the optical purity was calculated using the following relation:

$$po = \frac{[ {α}_{obs} ]}{[ {α}_{max} ]} \times 100 \%$$

where [α_{obs}] and [αₘₐₓ] denote, respectively, the optical activity of a mixture of enantiomers and that of one of the enantiomers in the pure state.

Numerically, the enantiomeric excess is equivalent to the optical purity, designated “po”, calculated after measuring the rotatory power of the mixture and comparing with the rotatory power of the dominant enantiomer.

The rotatory powers were measured in solution in chloroform at concentrations c expressed in g/100 mL.

“yield” signifies “chemical yield”.

“TLC” signifies “thin-layer chromatography”.

“Rf” signifies “retardation factor”.

“t_{R(min)}” signifies “retention time of the minor enantiomer”; “t_{R(dom)}” signifies “retention time of the dominant enantiomer”. They are expressed in minutes (min).

“ee” signifies “enantiomeric excess”.

$$po = \vert \frac{{[ α ]}_{obs}}{{[ α ]}_{max}} \vert \times 100 \%$$

### Synthesis of the Derivatives of Chiral α-Amino Acids

### General Procedure 1:

The following are introduced successively into a 90-mL tubular reactor under an argon atmosphere: 3 mmol of α-aminoacrylate, two equivalents of boronic acid RB(OH)₂, 1.5 mol % of dimer of chlorobis(ethylene)rhodium(I) [RhCl(═)₂]₂ (17.7 mg), 3.3 mol % of (S)-Difluorphos (69.7 mg) and one equivalent of NaHCO₃ (252 mg). The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography.

### General Procedure 2:

The following are introduced successively into a 10-mL dry tubular reactor with screw cap: 0.34 mmol of α-aminoacrylate, two equivalents of boronic ester RB(OR′)₂, 1.5 mol % dimer of chlorobis(ethylene)rhodium(I) [RhCl(═)₂]₂ (2.0 mg), 3.3 mol % of (S)-Difluorphos (7.7 mg), and one equivalent of NaHCO₃ (28.6 mg). The mixture is stirred under vacuum for about ten minutes and is then placed under argon. 1.4 mL of isopropanol is then introduced. After a succession of two vacuum/argon cycles, the reactor is immersed in a bath preheated to 60° C. After stirring for 20 h, if conversion is complete, the mixture is concentrated under vacuum; otherwise the mixture is heated to 70° C. Silica gel chromatography then gives the addition product.

### Example 1

### synthesis of (R)-methyl 2-tert-butoxycarbonylamino-3-phenylpropanoate

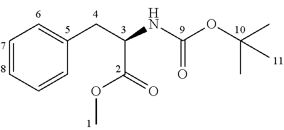 [SMILES: COC(=O)[C@@H](Cc1ccccc1)NC(=O)OC(C)(C)C]

The following are introduced into a tubular reactor under an argon atmosphere: 1 mmol of phenylboronic acid (122 mg) and 0.5 mmol of methyl 2-tert-butoxycarbonylaminoacrylate (100.5 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 8 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 98.3 mg of product is obtained (general procedure 1).
Colourless oil Yield=68% ee=98.8%

TLC: Rf=0.4 in ethyl acetate/cyclohexane eluent: 2/8

silica chromatography in ethyl acetate/cyclohexane eluent: 2/8

[α]_{D}²⁶=−48.3 (c=1; CHCl₃) for an enantiomeric excess of 98.8%.

HPLC: t_{R(min)}=67.3 min and t_{R(dom)}=72.8 min (Chiralpak AD-H, hexane/isopropanol: 99/1, 0.5 mL/min)

¹H NMR (CDCl₃, 300 MHz, δ): 1.41 (9H, s, H11), 3.03 (1H, dd, ²J=13.7 Hz, ³J=6.0 Hz, H4), 3.11 (1H, dd, ²J=13.7, ³J=6.0 Hz, H4′), 3.70 (3H, s, H1), 4.58 (1H, q app, ³J=6.0 Hz, H3), 5.01 (1H, d 1, ³J=7.8 Hz, NH), 7.11-7.31 (5H, m, H6, H7, H8).

¹³C NMR (CDCl₃, 75 MHz, δ): 28.3 (C11), 38.3 (C4), 52.2 (C1), 54.4 (C3), 79.9 (C10), 127.0 (C8), 128.5 (C6), 129.3 (C7), 136.0 (C5), 155.1 (C9), 172.3 (C2).

### Example 2

### synthesis of (R)-tert-butyl 2-tert-butoxycarbonylamino-3-phenylpropanoate

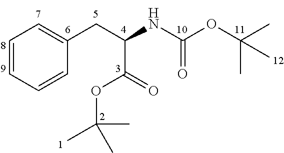 [SMILES: CC(C)(C)OC(=O)N[C@H](Cc1ccccc1)C(=O)OC(C)(C)C]

The following are introduced into a tubular reactor under an argon atmosphere: 1 mmol of phenylboronic acid (122 mg) and 0.5 mmol of tert-butyl 2-tert-butoxycarbonylaminoacrylate (121.8 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 8 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 124.9 mg of product is obtained (general procedure 1).
Colourless oil Yield=78% ee=99.8%

TLC: Rf=0.53 in ethyl acetate/cyclohexane eluent: 2/8

silica chromatography in ethyl acetate/cyclohexane eluent: 5/95

[α]_{D}²⁵=−22.8 (c=1; CHCl₃) for an enantiomeric excess of 99.8%

HPLC: t_{R(min)}=11.1 min and t_{R(dom)}=13.3 min (Chiralpak AS-H, hexane/isopropanol: 97/3, 0.5 mL/min)

¹H NMR (CDCl₃, 300 MHz, δ): 1.40 (9H, s, H1 or H12), 1.43 (9H, s, H1 or H12), 3.04-3.07, (2H, m, H5), 4.46 (1H, q app, ³J=7.7 Hz, H4), 5.02 (1H, d 1, ³J=7.7 Hz, NH), 7.17-7.32 (5H, m, H7, H8, H9).

¹³C NMR (CDCl₃, 75 MHz, δ): 26.9 (C1 or C12), 27.3 (C1 or C12), 37.5 (C5), 53.8 (C4), 78.6 (C2 or C11), 81.0 (C2 or C11), 125.8 (C9), 127.3 (C7), 128.5 (C8), 135.4 (C6), 154.1 (C10), 169.9 (C3).

### Example 3

### synthesis of (R)-isopropyl 2-benzyloxycarbonylamino-3-phenylpropanoate

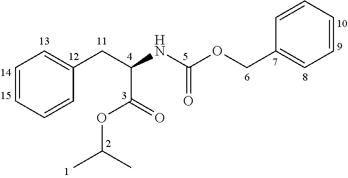 [SMILES: CC(C)OC(=O)[C@@H](Cc1ccccc1)NC(=O)OCc1ccccc1]

The following are introduced into a tubular reactor under an argon atmosphere: 1 mmol of phenylboronic acid (122 mg) and 0.5 mmol of isopropyl 2-benzyloxycarbonylaminoacrylate (181.5 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 8 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 63.4 mg of product is obtained (general procedure 1).
Yellow oil, Yield=37% ee=98.5%

TLC: Rf=0.41 in ethyl acetate/cyclohexane eluent: 1/9

silica chromatography in ethyl acetate/cyclohexane eluent: 15/85

[α]_{D}²⁵=−51.5 (c=1; CHCl₃) for an enantiomeric excess of 98.5%.

HPLC: t_{R(min)}=21.9 min and t_{R(dom)}=26.1 min (Chiralcel OJ, hexane/isopropanol: 95/5, 1 mL/min)

¹H NMR (CDCl₃, 300 MHz, δ): 1.18 (3H, d, ³J=6.5 Hz, H1), 1.20 (3H, d, ³J=6.5 Hz, H1′), 3.03-3.11 (2H, m, H11), 4.61 (1H, q app, ³J=6.2 Hz, H4), 4.92 (1H, hept, ³J=6.5 Hz, H2), 5.09 (2H, s, H6), 5.29 (1H, d 1, ³J=8.1 Hz, NH), 7.10-7.34 (10H, m, H8, H9, H10, H13, H14, H15).

¹³C NMR (CDCl₃, 75 MHz, δ): 21.7 (C1), 38.3 (C11), 54.9 (C4), 66.9 (C6), 69.3 (C2), 127.1, 128.1, 128.2, 128.5, 129.4 (C8, C9, C10, C13, C14, C15), 135.8 (C7 or C12), 136.3 (C7 or C12), 155.6 (C5), 171.0 (C3).

### Example 4

### synthesis of (R)-isopropyl 2-ethoxycarbonylamino-3-phenylpropanoate

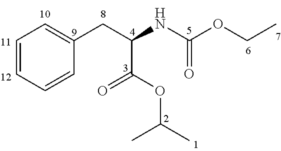 [SMILES: CCOC(=O)N[C@H](Cc1ccccc1)C(=O)OC(C)C]

The following are introduced into a tubular reactor under an argon atmosphere: 1 mmol of phenylboronic acid (122 mg) and 0.5 mmol of isopropyl 2-ethoxycarbonylaminoacrylate (150.5 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 8 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 193.4 mg of product is obtained (general procedure 1).
Colourless oil Yield=100% ee=97.9%

TLC: Rf=0.39 in ethyl acetate/cyclohexane eluent: 2/8

silica chromatography in ethyl acetate/cyclohexane eluent: 2/8

[α]_{D}²⁵=−27.0 (c=1; CHCl₃) for an enantiomeric excess of 97.9%.

HPLC: t_{R(min)}=25.5 min and t_{R(dom)}=30.9 min (Chiralcel OD-H, hexane/isopropanol: 99/1, 1 mL/min)

¹H NMR (CDCl₃, 300 MHz, δ): 1.19 (3H, t, ³J=6.6 Hz, H7), 1.21 (6H, d, ³J=6.3 Hz, H1), 3.07-3.09 (2H, m, H8), 4.10 (2H, q, ³J=6.6 Hz, H6), 4.58 (1H, q app, ³J=7.8 Hz, H4), 5.00 (1H, hept, ³J=6.3 Hz, H2), 5.15 (1H, d 1, ³J=7.8 Hz, NH), 7.13-7.16 (2H, m, H10), 7.21-7.31 (3H, m, H11, H12).

¹³C NMR (CDCl₃, 75 MHz, δ): 13.7 (C7), 20.8 (C1), 20.9 (C1′), 37.5 (C8), 53.9 (C4), 60.2 (C6), 68.4 (C2), 126.2 (C12), 127.6 (C10), 128.5 (C11), 135.1 (C9), 155.0 (C5), 170.3 (C3).

HRMS: Calculated for C₁₅H₂₁O₄NNa: 302.1363. Found: 302.1357.

### Example 5

### synthesis of (R)-isopropyl 2-tert-butoxycarbonylamino-3-(2-fluorophenyl)propanoate

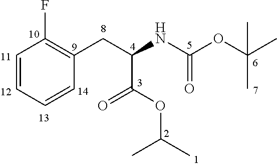 [SMILES: CC(C)OC(=O)[C@@H](Cc1ccccc1F)NC(=O)OC(C)(C)C]

The following are introduced into a tubular reactor under an argon atmosphere: 6 mmol of 2-fluorophenylboronic acid (840 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 867.8 mg of product is obtained (general procedure 1).
White solid MP=62° C. Yield=89% ee=99.3%

TLC: Rf=0.56 in ethyl acetate/cyclohexane eluent: 2/8

silica chromatography in ethyl acetate/cyclohexane eluent: from 2/98 to 10/90.

[α]_{D}²²=−38.1 (c=1; CHCl₃) for an enantiomeric excess of 99.3%.

HPLC: t_{R(min)}=9.0 min and t_{R(dom)}=10.3 min (Chiralpak AD-H, hexane/isopropanol: 90/10, 1 mL/min)

¹H NMR (CDCl₃, 300 MHz, δ): 1.18 (3H, d, ³J=6.2 Hz, H1), 1.22 (3H, d, ³J=6.2 Hz, H1′), 1.39 (9H, s, H7), 3.06 (1H, dd, ²J=14.4 Hz, ³J=6.6 Hz, H8), 3.16 (1H, dd, ²J=14.4 Hz, ³J=6.0 Hz, H8′), 4.52 (1H, q app, ³J=7.5 Hz, H4), 4.96-5.08 (2H, m, H2 and NH), 6.98-7.08 (2H, m, H13, H14), 7.15-7.24 (2H, m, H11, H12).

¹C NMR (CDCl₃, 75 MHz, δ): 21.5 (C1), 21.7 (C1′), 28.3 (C7), 31.9 (C8), 53.8 (C4), 69.2 (C2), 79.7 (C6), 115.3 (d, ²J_{C—F}=22.1 Hz, C11), 123.4 (d, ²J_{C—F}=15.9 Hz, C9), 124.0 (C13), 128.7 (d, ³J_{C—F}=8.0 Hz, C12), 131.7 (d, ³J_{C—F}=4.3 Hz, C14), 154.9 (C5), 161.4 (d, ¹J_{C—F}=245 Hz, C10), 171.2 (C3).

HRMS: Calculated for C₁₇H₂₄FNO₄Na: 348.1582. Found: 348.1584.

### Example 6

### synthesis of (R)-isopropyl 2-tert-butoxycarbonylamino-3-(4-fluorophenyl)propanoate

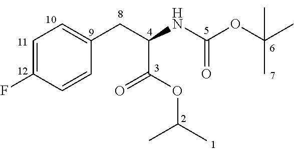 [SMILES: CC(C)OC(=O)[C@@H](Cc1ccc(F)cc1)NC(=O)OC(C)(C)C]

The following are introduced into a tubular reactor under an argon atmosphere: 6 mmol of 4-fluorophenylboronic acid (840 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 833.6 mg of product is obtained (general procedure 1).
White solid MP=73° C. Yield=85% ee=98.7%

TLC: Rf=0.38 in ethyl acetate/cyclohexane eluent: 2/8

silica chromatography in ethyl acetate/cyclohexane eluent: from 5/95 to 15/85.

[α]_{D}²⁸=−35.3 (c=1; CHCl₃) for an enantiomeric excess of 98.7%.

HPLC: t_{R(min)}=10.6 min and t_{R(dom)}=12.5 min (Chiralpak AD-H, hexane/isopropanol: 90/10, 1 mL/min)

¹H NMR (CDCl₃, 300 MHz, δ): 1.18 (3H, d, ³J=6.3 Hz, H1), 1.21 (3H, d, ³J=6.3 Hz, H1′), 1.41 (9H, s, H7), 3.03 (1H, dd, ²J=14.1 Hz, ³J=6.2 Hz, H8), 3.07 (1H, dd, ²J=14.1 Hz, ³J=6.2 Hz, H8′), 4.48 (1H, q app, ³J=7.5 Hz, H4), 4.95-5.03 (2H, m, H2 and NH), 6.93-7.03 (2H, m, H11), 7.08-7.15 (2H, m, H10).

¹³C NMR (CDCl₃, 75 MHz, δ): 21.69 (C1), 21.75 (C1′), 28.3 (C7), 37.6 (C8), 54.5 (C4), 69.2 (C2), 79.9 (C6), 115.2 (d, ²J_{C—F}=21.3 Hz, C11), 130.9 (³J_{C—F}=7.8 Hz, C10), 131.9 (C9), 155.0 (C5), 161.9 (d, ¹J_{C—F}=245 Hz, C12), 171.2 (C3).

### Example 7

### synthesis of (R)-isopropyl 2-tert-butoxycarbonylamino-3-(3-chlorophenyl)propanoate

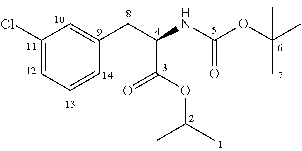 [SMILES: CC(C)OC(=O)[C@@H](Cc1cccc(Cl)c1)NC(=O)OC(C)(C)C]

The following are introduced into a tubular reactor under an argon atmosphere: 6 mmol of 3-chlorophenylboronic acid (939 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 926.8 mg of product is obtained (general procedure 1).
Yellow oil Yield=90% ee=99.7%

TLC: Rf=0.38 in ethyl acetate/cyclohexane eluent: 2/8

silica chromatography in ethyl acetate/cyclohexane eluent: from 5/95 to 15/85.

[α]_{D}²⁵=−41.1 (c=1; CHCl₃) for an enantiomeric excess of 99.7%.

HPLC: t_{R(min)}=5.1 min and t_{R(dom)}=5.6 min (Chiralpak IA, hexane/isopropanol: 90/10, 1 mL/min)

¹H NMR (CDCl₃, 300 MHz, δ): 1.21 (3H, d, ³J=6.0 Hz, H1), 1.23 (3H, d, ³J=6.0 Hz, H1′), 1.43 (9H, s, H7), 3.01 (1H, dd, ²J=13.8 Hz, ³J=6.0 Hz, H8), 3.09 (1H, dd, ²J=13.8 Hz, ³J=6.0 Hz, H8′), 4.50 (1H, q app, ³J=7.5 Hz, H4), 4.97-5.06 (2H, m, H2 and NH), 7.03-7.06 (1H, m, H13), 7.14 (1H, s, H10), 7.21-7.22 (m, 2H, H12, H14).

¹³C NMR (CDCl₃, 75 MHz, δ): 21.7 (C1), 28.3 (C7), 38.0 (C8), 54.4 (C4), 69.4 (C2), 79.9 (C6), 127.1 (C13 or C14), 127.6 (C13 or C14), 129.6 (C10, C12), 134.1 (C11), 138.3 (C9), 155.0 (C5), 171.2 (C3).

### Example 8

### synthesis of (R)-isopropyl 2-tert-butoxycarbonylamino-3-(3-bromophenyl)propanoate

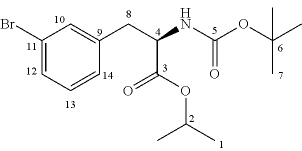 [SMILES: CC(C)OC(=O)[C@@H](Cc1cccc(Br)c1)NC(=O)OC(C)(C)C]

The following are introduced into a tubular reactor under an argon atmosphere: 6 mmol of 3-bromophenylboronic acid (1.205 g) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 208.8 mg of product is obtained (general procedure 1).
Beige solid MP=40° C. Yield=18% ee=96.2%

TLC: Rf=0.5 in ethyl acetate/cyclohexane eluent: 2/8

silica chromatography in ethyl acetate/cyclohexane eluent: 1/9

[α]_{D}²²=−38.5 (c=1; CHCl₃) for an enantiomeric excess of 96.2%.

HPLC: t_{R(min)}=13.7 min and t_{R(dom)}=15.8 min (Chiralpak AD-H, hexane/isopropanol: 95/5, 1 mL/min)

¹H NMR (CDCl₃, 300 MHz, δ): 1.24 (3H, d, ³J=6.3 Hz, H1), 1.27 (3H, d, ³J=6.3 Hz, H1′), 1.48 (9H, s, H7), 3.06-3.19 (2H, m, H8), 4.58 (1H, q app, ³J=7.8 Hz, H4), 5.02-5.10 (2H, m, H2 and NH), 7.19-7.22 (2H, m, H13, H14), 7.28-7.36 (2H, m, H10, H12).

¹³C NMR (CDCl₃, 75 MHz, δ): 21.7 (C1), 21.8 (C1′), 28.3 (C7), 38.4 (C8), 54.5 (C4), 69.1 (C2), 79.7 (C6), 126.9 (C13), 128.4 (C12, C14), 129.4 (C10, C11), 136.1 (C9), 155.1 (C5), 171.3 (C3).

HRMS: Calculated for C₁₇H₂₄O₄NNaBr: 408.0781 and 410.0761. Found: 408.0783 and 410.0762.

### Example 9

### synthesis of (R)-isopropyl 2-tert-butoxycarbonylamino-3-(4-bromophenyl)propanoate

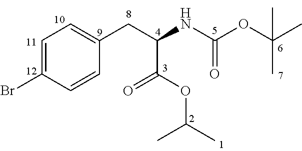 [SMILES: CC(C)OC(=O)[C@@H](Cc1ccc(Br)cc1)NC(=O)OC(C)(C)C]

The following are introduced into a tubular reactor under an argon atmosphere: 6 mmol of 4-bromophenylboronic acid (1.205 g) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 973.6 mg of product is obtained (general procedure 1).
Beige solid MP=82° C. Yield=84% ee=99.4%

TLC: Rf=0.53 in ethyl acetate/cyclohexane eluent: 2/8

silica chromatography in ethyl acetate/cyclohexane eluent: from 2/98 to 10/90.

[α]_{D}²⁰=−41.9 (c=1; CHCl₃) for an enantiomeric excess of 99.4%.

HPLC: t_{R(min)}=8.8 min and t_{R(dom)}=11.6 min (Chiralpak AD-H, hexane/isopropanol: 90/10, 1 mL/min)

¹H NMR (CDCl₃, 300 MHz, δ): 1.19 (3H, d, ³J=6.6 Hz, H1), 1.22 (3H, d, ³J=6.6 Hz, H1′), 1.42 (9H, s, H7), 2.99 (1H, dd, ²J=13.8 Hz, ³J=6.0 Hz, H8), 3.08 (1H, dd, ²J=13.8 Hz, ³J=6.0 Hz, H8′), 4.50 (1H, q app, ³J=7.5 Hz, H4), 4.96-5.04 (2H, m, H2 and NH), 7.03 (2H, d, ³J=8.2 Hz, H10), 7.41 (2H, d, ³J=8.2 Hz, H11).

¹³C NMR (CDCl₃, 75 MHz, δ): 21.7 (C1), 21.8 (C1′), 28.3 (C7), 37.8 (C8), 54.3 (C4), 69.3 (C2), 79.9 (C6), 120.9 (C12), 131.2 (C11), 131.5 (C10), 135.2 (C9), 155.0 (C5), 171.0 (C3).

HRMS: Calculated for C₁₇H₂₄BrNO₄Na: 408.0781 and 410.0763. Found: 408.0793 and 410.0768.

### Example 10

### synthesis of (R)-isopropyl 2-tert-butoxycarbonylamino-3-(3-chloro-4-fluorophenyl)propanoate

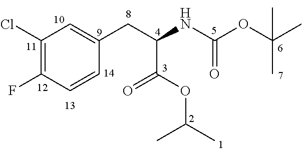 [SMILES: CC(C)OC(=O)[C@@H](Cc1ccc(F)c(Cl)c1)NC(=O)OC(C)(C)C]

The following are introduced into a tubular reactor under an argon atmosphere: 6 mmol of 3-chloro-4-fluorophenylboronic acid (1.047 g) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 947.6 mg of product is obtained (general procedure 1).
Yellow oil Yield=88% ee=99.9%

TLC: Rf=0.47 in ethyl acetate/cyclohexane eluent: 2/8

silica chromatography in ethyl acetate/cyclohexane eluent: 1/9.

[α]_{D}²⁰=−41.3 (c=1; CHCl₃) for an enantiomeric excess of 99.9%.

HPLC: t_{R(min)}=11.3 min and t_{R(dom)}=12.5 min (Chiralpak AD-H, hexane/isopropanol: 95/5, 1 mL/min)

¹H NMR (CDCl₃, 300 MHz, δ): 1.21 (3H, d, ³J=6.3 Hz, H1), 1.23 (3H, d, ³J=6.3 Hz, H1′), 1.42 (9H, s, H7), 2.97 (1H, dd, ²J=13.8 Hz, ³J=5.7 Hz, H8), 3.08 (1H, dd, ²J=13.8 Hz, ³J=6.0 Hz, H8′), 4.47 (1H, q app, ³J=7.5 Hz, H4), 4.97-5.08 (2H, m, H2 and NH), 7.00-7.08 (2H, m, H13, H14), 7.18 (1H, dd, ³J=7.1 and 1.5 Hz, H10).

¹³C NMR (CDCl₃, 75 MHz, δ): 21.7 (C1), 21.8 (C1′), 28.3 (C7), 37.4 (C8), 54.4 (C4), 69.5 (C2), 80.0 (C6), 116.4 (d, ²J_{C—F}=20.9 Hz, C13), 120.7 (d, ²J_{C—F}=18.6 Hz, C11), 129.1 (d, ³J_{C—F}=6.9 Hz, C10), 131.5 (C14), 133.3 (C9), 155.0 (C5), 157.2 (d, ¹J_{C—F}=248 Hz, C12), 170.9 (C3).

HRMS: Calculated for C₁₇H₂₃ClFNO₄Na: 382.1192 and 384.1163. Found: 382.1201 and 384.1172.

### Example 11

### (R)-isopropyl 2-tert-butoxycarbonylamino-3-(4-methoxycarbonylphenyl)propanoate

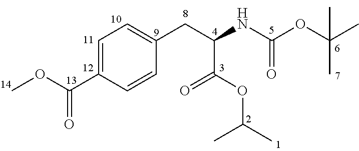 [SMILES: COC(=O)c1ccc(C[C@@H](NC(=O)OC(C)(C)C)C(=O)OC(C)C)cc1]

The following are introduced into a tubular reactor under an argon atmosphere: 4 mmol of 4-methoxycarbonylphenylboronic acid (720 mg) and 2 mmol of isopropyl 2-tert-butoxycarbonylaminoacrylate (466 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 8 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 185.9 mg of product is obtained (general procedure 1).
Colourless oil Yield=25% ee=99.9%

TLC: Rf=0.32 in ethyl acetate/cyclohexane eluent: 2/8

silica chromatography in ethyl acetate/cyclohexane eluent: 1/9.

[α]_{D}²⁵=−36.4 (c=1; CHCl₃) for an enantiomeric excess of 99.9%.

HPLC: t_{R(min)}=13.9 min and t_{R(dom)}=18.6 min (Chiralpak IA, hexane/isopropanol: 95/5, 1 mL/min)

¹H NMR (CDCl₃, 300 MHz, δ): 1.18 (3H, d, ³J=6.3 Hz, H1), 1.21 (3H, d, ³J=6.3 Hz, H1′), 1.41 (9H, s, H7), 3.08 (1H, dd, ²J=13.6 Hz, ³J=6.0 Hz, H8), 3.17 (1H, dd, ²J=13.6 Hz, ³J=6.0 Hz, H8′), 3.89 (3H, s, H14), 4.53 (1H, q app, ³J=7.5 Hz, H4), 4.95-5.03 (2H, m, H2; NH), 7.22 (2H, d, ³J=8.4 Hz, H10), 7.95 (2H, d, ³J=8.1 Hz, H11).

¹³C NMR (CDCl₃, 75 MHz, δ): 21.7 (C1), 28.3 (C7), 38.4 (C8), 52.1 (C4), 54.3 (C14), 69.4 (C2), 79.9 (C6), 128.8 (C12), 129.5 (C10), 129.7 (C11), 141.7 (C9), 155.0 (C5), 166.9 (C13), 171.0 (C3). HRMS: Calculated for C₁₉H₂₇O₆NNa: 388.1731. Found: 388.1734.

### Example 12

### (R)-isopropyl 2-tert-butoxycarbonylamino-3-(4-trifluoromethylphenyl)propanoate

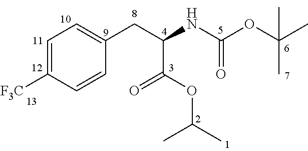 [SMILES: CC(C)OC(=O)[C@@H](Cc1ccc(C(F)(F)F)cc1)NC(=O)OC(C)(C)C]

The following are introduced into a tubular reactor under an argon atmosphere: 1 mmol of 4-trifluoromethylphenylboronic acid (190 mg) and 0.5 mmol of isopropyl 2-tert-butoxycarbonylaminoacrylate (114.7 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 4 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 120.8 mg of product is obtained (general procedure 1).
White solid MP=81° C. Yield=65% ee=99.4%

TLC: Rf=0.42 in ethyl acetate/cyclohexane eluent: 2/8

silica chromatography in ethyl acetate/cyclohexane eluent: 5/95.

[α]_{D}²⁵=−38.5 (c=1; CHCl₃) for an enantiomeric excess of 99.4%.

HPLC: t_{R(min)}=9.3 min and t_{R(dom)}=12.4 min (Chiralpak AD-H, hexane/isopropanol: 95/5, 1 mL/min)

¹H NMR (CDCl₃, 300 MHz, δ): 1.17 (3H, d, ³J=6.3 Hz, H1), 1.20 (3H, d, ³J=6.3 Hz, H1′), 1.40 (9H, s, H7), 3.06 (1H, dd, ²J=13.7 Hz, ³J=6.2 Hz, H8), 3.17 (1H, dd, ²J=13.7 Hz, ³J=6.2 Hz, H8′), 4.53 (1H, q app, ³J=7.2 Hz, H4), 4.95-5.07 (2H, m, H2; NH), 7.27 (2H, d, ³J=8.1 Hz, H10), 7.53 (2H, d, ³J=8.1 Hz, H11).

¹³C NMR (CDCl₃, 75 MHz, δ): 20.6 (C1), 20.7 (C1′), 27.2 (C7), 37.3 (C8), 53.3 (C4), 68.4 (C2), 79.0 (C6), 123.2 (q, ¹J_{C—F}=272 Hz, C13), 124.2 (C11), 128.2 (q, ²J_{C—F}=32.0 Hz, C12), 128.8 (C10), 139.5 (C9), 154.0 (C5), 169.9 (C3).

HRMS: Calculated for C₁₈H₂₄O₄NF₃Na: 398.1550. Found: 398.1555.

### Example 13

### (R)-isopropyl 2-tert-butoxycarbonylamino-3-(3-acetylphenyl)propanoate

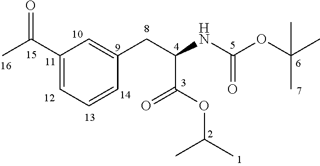 [SMILES: CC(=O)c1cccc(C[C@@H](NC(=O)OC(C)(C)C)C(=O)OC(C)C)c1]

The following are introduced into a tubular reactor under an argon atmosphere: 4 mmol of 3-acetylphenylboronic acid (656 mg) and 2 mmol of isopropyl 2-tert-butoxycarbonylaminoacrylate (466 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 4 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 639.6 mg of product is obtained (general procedure 1).
Yellow oil Yield=91% ee=98.8%

TLC: Rf=0.29 in ethyl acetate/cyclohexane eluent: 2/8

silica chromatography in ethyl acetate/cyclohexane eluent: from 5/95 to 20/80.

[α]_{D}²⁸=−47.3 (c=1; CHCl₃) for an enantiomeric excess of 98.8%.

HPLC: t_{R(min)}=15.9 min and t_{R(dom)}=22.0 min (Chiralpak AD-H, hexane/isopropanol: 90/10, 1 mL/min)

¹H NMR (CDCl₃, 300 MHz, δ): 1.20 (3H, d, ³J=6.0 Hz, H1), 1.21 (3H, d, ³J=6.0 Hz, H1′), 1.40 (9H, s, H7), 2.6 (3H, s, H16), 3.08 (1H, dd, ²J=15.0 Hz, ³J=6.0 Hz, H8) 3.19 (1H, dd, ²J=15.0 Hz, ³J=6.0 Hz, H8′), 4.55 (1H, q app, ³J=6.0 Hz, H4), 4.93-5.05 (2H, m, H2 and NH), 7.34-7.41 (2H, m, H13, H14), 7.74 (1H, s, H10), 7.80-7.83 (1H, m, H12).

¹³C NMR (CDCl₃, 75 MHz, δ): 21.7 (C1), 21.8 (C1′), 26.6 (C16), 28.3 (C7), 38.2 (C8), 54.4 (C4), 69.4 (C2), 79.9 (C6), 127.0 (C12), 128.7 (C10), 129.3 (C13), 134.2 (C14), 136.9 (C11), 137.2 (C9), 154.9 (CS), 171.0 (C3), 197.9 (C15).

HRMS: Calculated for C₁₉H₂₇NO₅Na: 372.1784. Found: 372.1786.

### Example 14

### (R)-isopropyl 2-tert-butoxycarbonylamino-3-(3-nitrophenyl)propanoate

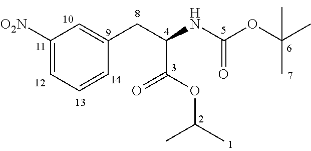 [SMILES: CC(C)OC(=O)[C@@H](Cc1cccc([N+](=O)[O-])c1)NC(=O)OC(C)(C)C]

The following are introduced into a tubular reactor under an argon atmosphere: 5 mmol of 3-nitrophenylboronic acid (835 mg) and 2.5 mmol of isopropyl 2-tert-butoxycarbonylaminoacrylate (585 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 10 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 599.4 mg of product is obtained (general procedure 1).
Yellow solid MP=56° C. Yield=68% ee=99.9%

TLC: Rf=0.29 in ethyl acetate/heptane eluent: 2/8

silica chromatography in ethyl acetate/heptane eluent: 1/9.

[α]_{D}²⁸=−41.0 (c=1; CHCl₃) for an enantiomeric excess of 99.9%.

HPLC: t_{R(min)}=10.4 min and t_{R(dom)}=11.6 min (Chiralpak IA, hexane/isopropanol: 95/5, 1 mL/min) ¹H NMR (CDCl₃, 300 MHz, δ): 1.24 (6H, d, ³J=6.3 Hz, H1), 1.42 (9H, s, H7), 3.13 (1H, dd, ²J=13.6 Hz, ³J=6.0 Hz, H8), 3.28 (1H, dd, ²J=13.6 Hz, ³J=5.7 Hz, H8′), 4.55 (1H, q app, ³J=6.6 Hz, H4), 4.99-5.11 (2H, m, H2; NH), 7.44-7.53 (2H, m, H13, H14), 8.04 (1H, s, H10), 8.11 (1H, td, ³J=7.5 and 1.8 Hz, H12).

¹³C NMR (CDCl₃, 75 MHz, δ): 21.8 (C1), 28.2 (C7), 38.1 (C8), 54.3 (C4), 69.8 (C2), 80.2 (C6), 122.1 (C12), 124.4 (C10), 129.3 (C13), 135.7 (C14), 138.5 (C9), 148.2 (C11). 154.9 (C5), 170.6 (C3).

HRMS: Calculated for C₁₇H₂₄O₆N₂Na: 375.1527. Found: 375.1529.

### Example 15

### (R)-isopropyl 2-tert-butoxycarbonylamino-3-(3-trifluoromethylphenyl)propanoate

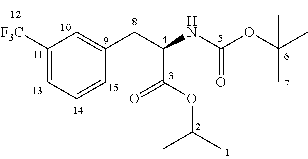 [SMILES: CC(C)OC(=O)[C@@H](Cc1cccc(C(F)(F)F)c1)NC(=O)OC(C)(C)C]

The following are introduced into a tubular reactor under an argon atmosphere: 6 mmol of 3-trifluoromethylphenylboronic acid (1.14 g) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 845.3 mg of product is obtained (general procedure 1).
Beige solid MP=48° C. Yield=75% ee=99.0%

TLC: Rf=0.47 in ethyl acetate/cyclohexane eluent: 2/8

silica chromatography in ethyl acetate/cyclohexane eluent: 1/9.

[α]_{D}²⁵=−56.4 (c=1; CHCl₃) for an enantiomeric excess of 99.0%.

HPLC: t_{R(min)}=4.3 min and t_{R(dom)}=4.6 min (Chiralpak IA, hexane/isopropanol: 90/10, 1 mL/min)

¹H NMR (CDCl₃, 300 MHz, δ): 1.18 (3H, d, ³J=6.3 Hz, H1), 1.21 (3H, d, ³J=6.3 Hz, H1′), 1.42 (9H, s, H7), 3.10 (1H, dd, ²J=13.8 Hz, ³J=5.7 Hz, H8), 3.19 (1H, dd, ²J=13.8 Hz, ³J=6.0 Hz, H8′), 4.53 (1H, q app, ³J=7.2 Hz, H4), 4.97-5.08 (2H, m, H2 and NH), 7.34-7.51 (4H, H10, H13, H14, H15).

¹³C NMR (CDCl₃, 75 MHz, δ): 21.6 (C1), 21.7 (C1′), 28.2 (C7), 38.2 (C8), 54.3 (C4), 69.5 (C2), 79.9 (C6), 123.8 (C13), 124.1 (q, ¹J_{C—F}=272 Hz, C12), 126.2 (C10), 128.8 (C14), 130.7 (q, ²J_{C—F}=32.1 Hz, C11), 132.8 (C15), 137.3 (C9), 154.9 (C5), 170.9 (C3).

HRMS: Calculated for C₁₈H₂₄O₄NF₃Na: 398.1550. Found: 398.1555.

### Example 16

### (R)-isopropyl 2-tert-butoxycarbonylamino-3-(3-acetamidophenyl)propanoate

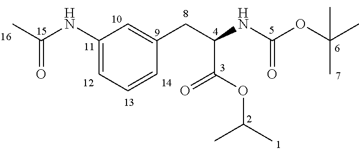 [SMILES: CC(=O)Nc1cccc(C[C@@H](NC(=O)OC(C)(C)C)C(=O)OC(C)C)c1]

The following are introduced into a tubular reactor under an argon atmosphere: 4 mmol of 3-acetamidophenylboronic acid (716 mg) and 2 mmol of isopropyl 2-tert-butoxycarbonylaminoacrylate (466 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 8 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 501.5 mg of product is obtained (general procedure 1).
White solid MP=110° C. Yield=54% ee=98.3%

TLC: Rf=0.18 in acetone/dichloromethane eluent 1/9

silica chromatography in acetone/dichloromethane eluent: from 2/98 to 1/9.

[α]_{D}²¹=−32.4 (c=1; CHCl₃) for an enantiomeric excess of 98.3%.

HPLC: t_{R(min)}=19.7 min and t_{R(dom)}=22.7 min (Chiralpak AD-H, hexane/isopropanol: 90/10, 1 mL/min)

¹H NMR (CDCl₃, 300 MHz, δ): 1.18 (3H, d, ³J=6.0 Hz, H1), 1.20 (3H, d, ³J=6.0 Hz, H1′), 1.40 (9H, s, H7), 2.14 (3H, s, H16), 2.94-3.09 (2H, m, H8), 4.47 (1H, q app, ³J=6.9 Hz, H4), 4.96-5.04 (2H, m, H2 and NH), 6.88 (1H, d, ³J=7.5 Hz, H14), 7.21 (1H, t, ³J=7.8 Hz, H13), 7.30 (1H, s, H10), 7.40 (1H, d, ³J=8.1 Hz, H12), 7.53 (1H, s 1, NH).

¹³C NMR (CDCl₃, 75 MHz, δ): 21.6 (C1), 21.7 (C1′), 24.6 (C16), 28.3 (C7), 38.3 (C8), 54.5 (C4), 69.2 (C2), 79.9 (C6), 118.4 (C12), 120.6 (C10), 125.2 (C14), 129.0 (C13), 137.1 (C11), 138.2 (C9), 155.2 (C5), 168.4 (C15), 171.4 (C3).

HRMS: Calculated for C₁₉H₂₈O₅N₂Na: 387.1890. Found: 387.1894.

### Example 17

### (R)-isopropyl 2-tert-butoxycarbonylamino-3-(3-tert-butoxycarbonylaminophenyl)propanoate

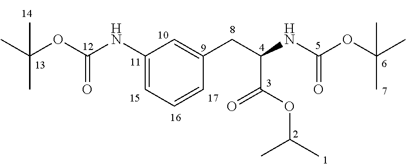 [SMILES: CC(C)OC(=O)[C@@H](Cc1cccc(NC(=O)OC(C)(C)C)c1)NC(=O)OC(C)(C)C]

The following are introduced into a tubular reactor under an argon atmosphere: 2.4 mmol of 3-tert-butoxycarbonylaminophenylboronic acid (600 mg) and 1.2 mmol of isopropyl 2-tert-butoxycarbonylaminoacrylate (282.2 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 6 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 401.8 mg of product is obtained (general procedure 1).
White solid MP=111° C. Yield=79% ee=99.9%

TLC: Rf=0.27 in ethyl acetate/cyclohexane eluent: 2/8

silica chromatography in ethyl acetate/cyclohexane eluent: 2/98 to 15/85.

[α]_{D}²²=−28.7 (c=1; CHCl₃) with an enantiomeric excess of 99.9%.

HPLC: t_{R(min)}=11.1 min and t_{R(dom)}=19.2 min (Chiralpak IA, hexane/isopropanol: 95/5, 1 mL/min)

¹H NMR (CDCl₃, 300 MHz, δ): 1.20 (3H, d, ³J=6.3 Hz, H1), 1.21 (3H, d, ³J=6.0 Hz, H1′), 1.41 (9H, s, H7), 1.50 (9H, s, H14), 2.98 (1H, dd, ²J=13.9 Hz, ³J=6.6 Hz, H8), 3.06 (1H, dd, ²J=13.9 Hz, ³J=6.0 Hz, H8′), 4.48 (1H, q app, ³J=7.8 Hz, H4), 4.96-5.05 (2H, m, H2 and NH), 6.52 (1H, s 1, NH), 6.52-6.84 (1H, m, H17), 7.15-7.26 (3H, m, H10, H15, H16).

¹³C NMR (CDCl₃, 75 MHz, δ): 21.7 (C1), 21.8 (C1′), 28.3 (C7, C14), 38.3 (C8), 54.4 (C4), 69.1 (C2), 79.7 (C6), 80.4 (C13), 117.1 (C15), 119.4 (C10), 124.0 (C17), 129.0 (C16), 137.1 (C11), 138.5 (C9), 152.6 (C12), 155.1 (C5), 171.3 (C3).

HRMS: Calculated for C₂₂H₃₄N₂O₆Na: 445.2309. Found: 445.2317.

### Example 18

### (R)-isopropyl 2-tert-butoxycarbonylamino-3-(3-methylthiophenyl)propanoate

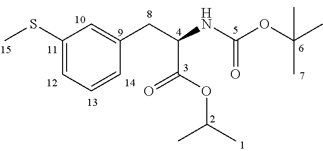 [SMILES: CSc1cccc(C[C@@H](NC(=O)OC(C)(C)C)C(=O)OC(C)C)c1]

The following are introduced into a tubular reactor under an argon atmosphere: 5 mmol of 3-thiomethylphenylboronic acid (840 mg) and 2.5 mmol of isopropyl 2-tert-butoxycarbonylaminoacrylate (585 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 10 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 782.8 mg of product is obtained (general procedure 1).
White solid MP=68° C. Yield=88% ee=99.6%

TLC: Rf=0.36 in ethyl acetate/heptane eluent: 2/8

silica chromatography in ethyl acetate/heptane eluent: 5/95.

[α]_{D}²⁶=−39.1 (c=1; CHCl₃) for an enantiomeric excess of 99.6%.

HPLC: t_{R(min)}=8.4 min and t_{R(dom)}=9.6 min (Chiralpak IA, hexane/isopropanol: 95/5, 1 mL/min)

¹H NMR (CDCl₃, 300 MHz, δ): 1.20 (3H, d, ³J=6.6 Hz, H1), 1.22 (3H, d, ³J=6.3 Hz, H1′), 1.42 (9H, s, H7), 2.46 (3H, s, H15), 2.97-3.11 (2H, m, H8), 4.51 (1H, q app, ³J=7.8 Hz, H4), 4.96-5.05 (2H, m, H2, NH), 6.92 (1H, d, ³J=7.5 Hz, H14), 7.02 (1H, s, H10), 7.11-7.22 (2H, m, H12, H13). ¹³C NMR (CDCl₃, 75 MHz, δ): 15.7 (C15), 21.7 (C1), 21.8 (C1′), 28.3 (C7), 38.2 (C8), 54.4 (C4), 69.2 (C2), 79.8 (C6), 125.0 (C10), 126.1 (C14), 127.5 (C12), 128.8 (C13), 136.9 (C11), 138.6 (C9), 155.0 (C5), 171.2 (C3).

HRMS: Calculated for C₁₈H₂₇O₄NNaS: 376.1553. Found: 376.1557.

### Example 19

### (R)-isopropyl 2-tert-butoxycarbonylamino-3-(3-methoxyphenyl)propanoate

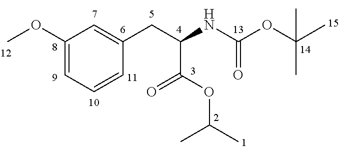 [SMILES: COc1cccc(C[C@@H](NC(=O)OC(C)(C)C)C(=O)OC(C)C)c1]

The following are introduced into a tubular reactor under an argon atmosphere: 6 mmol of 3-methoxyphenylboronic acid (912 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 905.3 mg of product is obtained (general procedure 1).
Yellow oil Yield=90% ee=98.9%

TLC: Rf=0.36 in ethyl acetate/cyclohexane eluent: 1/9

silica chromatography in ethyl acetate/cyclohexane eluent: 1/9

[α]_{D}²²=−56.5 (c=0.99; CHCl₃) for an enantiomeric excess of 98.9%.

HPLC: t_{R(min)}=14.1 min and t_{R(dom)}=17.6 min (Chiralpak AD-H, hexane/isopropanol: 95/5, 1 mL/min)

¹H NMR (CDCl₃, 300 MHz, δ): 1.19 (3H, d, ³J=6.2 Hz, H1), 1.22 (3H, d, ³J=6.2 Hz, H1′), 1.42 (9H, s, H15), 2.93-3.12 (2H, m, H5), 3.77 (1H, s, H12), 4.43 (1H, q app, ³J=7.8 Hz, H4), 4.96-5.02 (2H, m, H2 and NH), 6.67-6.79 (3H, m, H7, H9, H11), 7.19 (1H, t, ³J=7.9 Hz, H10).

¹³C NMR (CDCl₃, 75 MHz, δ): 21.6 (C1), 21.7 (C1′), 28.3 (C15), 38.3 (C5), 54.4 (C4), 55.1 (C12), 69.1 (C2), 79.8 (C14), 112.4 (C9), 115.1 (C7), 121.8 (C11), 129.4 (C10), 137.6 (C6), 155.1 (C13), 159.6 (C8), 171.3 (C3).

### Example 20

### (R)-isopropyl 2-tert-butoxycarbonylamino-3-(3-hydroxyphenyl)propanoate

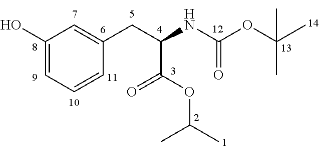 [SMILES: CC(C)OC(=O)[C@@H](Cc1cccc(O)c1)NC(=O)OC(C)(C)C]

The following are introduced into a tubular reactor under an argon atmosphere: 1 mmol of 3-hydroxyphenylboronic acid (138 mg) and 0.5 mmol of isopropyl 2-tert-butoxycarbonylaminoacrylate (114.7 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 2 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 79 mg of product is obtained (general procedure 1).
Yellow oil Yield=49% ee=96.5%

TLC: Rf=0.37 in ethyl acetate/cyclohexane eluent: 2/8

silica chromatography in ethyl acetate/cyclohexane eluent: 2/8

[α]_{D}²⁶=+69.7 (c=1; CHCl₃) for an enantiomeric excess of 96.5%.

HPLC: t_{R(min)}=13.8 min and t_{R(dom)}=16.3 min (Chiralpak AS-H, hexane/isopropanol: 90/10, 0.5 mL/min)

¹H NMR (CDCl₃, 300 MHz, δ): 1.18 (3H, d, ³J=6.3 Hz, H1), 1.20 (3H, d, ³J=6.3 Hz, H1′), 1.41 (9H, s, H14), 2.89-3.11 (2H, m, H5), 4.50 (1H, q app, ³J=7.8 Hz, H4), 4.97-5.09 (2H, m, H2 and NH), 6.54 (1H, s 1, OH), 6.67-6.73 (3H, m, H7, H9, H11), 7.11 (1H, t, ³J=8.1 Hz, H10).

¹³C NMR (CDCl₃, 75 MHz, δ): 21.6 (C1), 21.7 (C1′), 28.3 (C14), 38.2 (C5), 54.5 (C4), 69.3 (C2), 80.1 (C13), 114.1 (C9), 116.3 (C7), 121.3 (C11), 129.6 (C10), 137.6 (C6), 155.4 (C12), 156.3 (C8), 171.6 (C3).

HRMS: Calculated for C₁₇H₂₅O₅NNa: 346.1625. Found: 346.1625.

### Example 21

### (R)-isopropyl 2-tert-butoxycarbonylamino-3-(3-trifluoromethoxyphenyl)propanoate

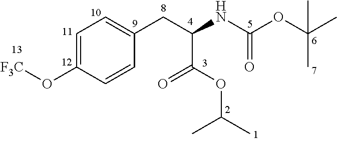 [SMILES: CC(C)OC(=O)[C@@H](Cc1ccc(OC(F)(F)F)cc1)NC(=O)OC(C)(C)C]

The following are introduced into a tubular reactor under an argon atmosphere: 3 mmol of 3-trifluoromethoxyphenylboronic acid (618 mg) and 1.5 mmol of isopropyl 2-tert-butoxycarbonylaminoacrylate (353 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 6 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 575.2 mg of product is obtained (general procedure 1).
White solid MP=75° C. Yield=98% ee=99.9%

TLC: Rf=0.44 in ethyl acetate/heptane eluent: 2/8

silica chromatography in ethyl acetate/heptane eluent: 5/95

[α]_{D}²⁸=−38.7 (c=1; CHCl₃) for an enantiomeric excess of 99.9%.

HPLC: t_{R(min)}=7.3 min and t_{R(dom)}=8.5 min (Chiralpak AS-H, hexane/isopropanol: 90/10, 0.5 mL/min)

¹H NMR (CDCl₃, 300 MHz, δ): 1.17 (3H, d, ³J=6.3 Hz, H1), 1.21 (3H, d, ³J=6.3 Hz, H1′), 1.41 (9H, s, H7), 3.04 (1H, dd, ²J=13.7 Hz, ³J=6.3 Hz, H8), 3.11 (1H, dd, ²J=13.7 Hz, ³J=6.3 Hz, H8′), 4.51 (1H, q app, ³J=7.2 Hz, H4), 4.93-5.04 (2H, m, H2, NH), 7.11-7.20 (4H, m, H10, H11). ¹³C NMR (CDCl₃, 75 MHz, δ): 21.6 (C1), 21.7 (C1′), 28.2 (C7), 37.9 (C8), 54.4 (C4), 69.3 (C2), 79.9 (C6), 120.4 (q, ¹J_{C—F}=257 Hz, C13), 120.9 (C11), 130.8 (C10), 135.0 (C9), 148.2 (C12), 155.0 (C5), 171.1 (C3).

HRMS: Calculated for C₁₈H₂₄O₅NF₃Na: 414.1499. Found: 414.1492.

### Example 22

### (R)-isopropyl 2-tert-butoxycarbonylamino-3-(4-methoxyphenyl)propanoate

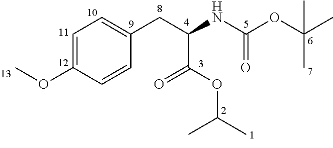 [SMILES: COc1ccc(C[C@@H](NC(=O)OC(C)(C)C)C(=O)OC(C)C)cc1]

The following are introduced into a tubular reactor under an argon atmosphere: 6 mmol of 4-methoxyphenylboronic acid (912 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 769.2 mg of product is obtained (general procedure 1).
White solid MP=70° C. Yield=76% ee=99%

TLC: Rf=0.42 in ethyl acetate/cyclohexane eluent: 2/8

silica chromatography in ethyl acetate/cyclohexane eluent: from 5/95 to 1/9.

[α]_{D}²⁶=−37.4 (c=1; CHCl₃) for an enantiomeric excess of 99%.

HPLC: t_{R(min)}=7.1 min and t_{R(dom)}=8.3 min (Chiralpak AS-H, hexane/isopropanol: 95/5, 1 mL/min)

¹H NMR (CDCl₃, 300 MHz, δ): 1.20 (3H, d, ³J=6.0 Hz, H1), 1.21 (3H, d, ³J=6.0 Hz, H1′), 1.41 (9H, s, H7), 2.94-3.07 (2H, m, H8), 3.77 (3H, s, H15), 4.46 (1H, q app, ³J=7.8 Hz, H4), 4.96-5.04 (2H, m, H2, NH), 6.80-6.83 (2H, m, H11), 7.04-7.07 (2H, m, H10).

¹³C NMR (CDCl₃, 75 MHz, δ): 21.7 (C1), 21.8 (C1′), 28.3 (C7), 37.4 (C8), 54.6 (C4), 55.2 (C13), 69.0 (C2), 79.7 (C6), 113.9 (C11), 128.1 (C9), 130.4 (C10), 155.1 (C5), 158.6 (C12), 171.4 (C3).

### Example 23

### (R)-isopropyl 2-tert-butoxycarbonylamino-3-(4-dimethylaminophenyl)propanoate

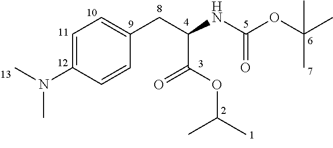 [SMILES: CC(C)OC(=O)[C@@H](Cc1ccc(N(C)C)cc1)NC(=O)OC(C)(C)C]

The following are introduced into a tubular reactor under an argon atmosphere: 5 mmol of 4-dimethylaminophenylboronic acid (825 mg) and 2.5 mmol of isopropyl 2-tert-butoxycarbonylaminoacrylate (585 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 10 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 260.2 mg of product is obtained (general procedure 1).
Yellow oil Yield=30% ee=99.5%

TLC: Rf=0.3 in ethyl acetate/heptane eluent: 2/8

silica chromatography in ethyl acetate/heptane eluent: 1/9

[α]_{D}²⁸=−59.5 (c=1; CHCl₃) for an enantiomeric excess of 99.5%.

HPLC: t_{R(min)}=9.0 min and t_{R(dom)}=10.0 min (Chiralpak AS-H, hexane/isopropanol: 95/5, 1 mL/min)

¹H NMR (CDCl₃, 300 MHz, δ): 1.22 (3H, d, ³J=6.3 Hz, H1), 1.23 (3H, d, ³J=6.0 Hz, H1′), 1.42 (9H, s, H7), 2.91 (6H, s, H13), 2.94-3.03 (2H, m, H8), 4.45 (1H, q app, ³J=8.1 Hz, H4), 4.93-5.06 (2H, m, H2, NH), 6.66 (2H, d, ³J=8.7 Hz, H11), 6.99 (2H, d, ³J=8.7 Hz, H10).

¹³C NMR (CDCl₃, 75 MHz, δ): 21.7 (C1), 21.8 (C1′), 28.3 (C7), 37.2 (C8), 40.7 (C13), 54.7 (C4), 68.9 (C2), 79.6 (C6), 112.8 (C11), 130.1 (C9, C10), 149.6 (C12), 155.2 (C5), 171.6 (C3).

HRMS: Calculated for C₁₉H₃₁O₄N₂: 351.2278. Found: 351.2281.

### Example 24

### (R)-isopropyl 3-(benzo[d][1,3]dioxol-5-yl)-2-(tert-butoxycarbonylamino)propanoate

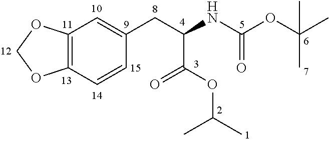 [SMILES: CC(C)OC(=O)[C@@H](Cc1ccc2c(c1)OCO2)NC(=O)OC(C)(C)C]

The following are introduced into a tubular reactor under an argon atmosphere: 6 mmol of 1-benzofuran-2-ylboronic acid (1.000 g) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 931.1 mg of product is obtained (general procedure 1).
White solid MP=74° C. Yield=88% ee=99.3%

TLC: Rf=0.47 in ethyl acetate/cyclohexane eluent: 2/8

silica chromatography in ethyl acetate/cyclohexane eluent: from 2/98 to 1/9.
[α]_{D}²³=−38.3 (c=1; CHCl₃) for an enantiomeric excess of 99.3%.

HPLC: t_{R(min)}=8.3 min and t_{R(dom)}=9.9 min (Chiralpak AS-H, hexane/isopropanol: 95/5, 1 mL/min)

¹H NMR (CDCl₃, 300 MHz, δ): 1.20 (3H, d, ³J=6.3 Hz, H1) 1.22 (3H, d, ³J=6.3 Hz, H1′), 1.41 (9H, s, H7), 2.91-3.04 (2H, m, H8), 4.44 (1H, q app, ³J=7.8 Hz, H4), 4.96-5.04 (2H, m, H2 and NH), 5.87-5.90 (1H, m, H12), 6.57 (1H, dd, ³J=8.0 Hz, J=1.6 Hz, H15), 6.62 (1H, d, J=1.6 Hz, H10), 6.70 (1H, d, ³J=8.0 Hz, H14).

¹³C NMR (CDCl₃, 75 MHz, δ): 21.7 (C1), 21.8 (C1′), 28.3 (C7), 38.0 (C8), 54.6 (C4), 69.1 (C2), 79.7 (C6), 100.9 (C12), 108.2 (C14), 109.7 (C10), 122.5 (C15), 129.7 (C9), 146.5 (C13), 147.6 (C11), 155.1 (C5), 171.3 (C3).

HRMS: Calculated for C₁₈H₂₅NO₆Na: 374.1574. Found: 374.1578.

### Example 25

### (R)-isopropyl 2-tert-butoxycarbonylamino-3-(thiophen-3-yl)propanoate

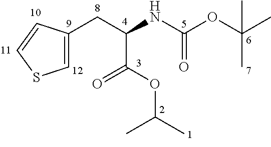 [SMILES: CC(C)OC(=O)[C@@H](Cc1ccsc1)NC(=O)OC(C)(C)C]

The following are introduced into a tubular reactor under an argon atmosphere: 6 mmol of 3-thiopheneboronic acid (768 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 368.9 mg of product is obtained (general procedure 1).
Colourless oil Yield=39% ee=99.3%

TLC: Rf=0.40 in ethyl acetate/heptane eluent: 2/8

silica chromatography in ethyl acetate/heptane eluent: 1/9.
[α]_{D}^{22.5}=−33.5 (c=1.012; CHCl₃) for an enantiomeric excess of 99.3%.

HPLC: t_{R(min)}=8.1 min and t_{R(dom)}=9.0 min (Chiralpak IA, hexane/isopropanol: 95/5, 1 mL/min)

¹H NMR (CDCl₃, 300 MHz, δ): 1.21 (3H, d, ³J=6.3 Hz, H1), 1.24 (3H, d, ³J=6.6 Hz, H1′), 1.44 (9H, s, H7), 3.06-3.19 (2H, m, H8), 4.51 (1H, q app, ³J=7.8 Hz, H4), 4.98-5.08 (2H, m, H2, NH), 6.91 (1H, dd, ³J=4.8 Hz, ⁴J=1.2 Hz, H10), 7.01 (1H, d 1, ⁴J=1.8 Hz, H12), 7.26 (1H, dd, ³J=4.8 Hz, ⁴J=1.8 Hz, H11).

¹³C NMR (CDCl₃, 75 MHz, δ): 21.7 (C1), 28.3 (C7), 32.8 (C8), 54.0 (C4), 69.1 (C2), 79.8 (C6), 122.7 (C10), 125.7 (C11), 128.5 (C12), 136.3 (C9), 155.1 (C5), 171.3 (C3).

HRMS: Calculated for C₁₅H₂₃O₄NNaS: 336.1240. Found: 336.1238.

### Example 26

### (R)-isopropyl 2-tert-butoxycarbonylamino-3-(thiophen-2-yl)propanoate

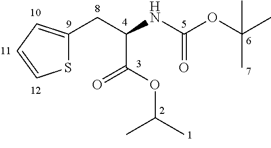 [SMILES: CC(C)OC(=O)[C@@H](Cc1cccs1)NC(=O)OC(C)(C)C]

The following are introduced into a tubular reactor under an argon atmosphere: 6 mmol of 2-thiopheneboronic acid (791 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 34.5 mg of product is obtained (general procedure 1).
Yellow oil Yield=4% ee=98.7%

TLC: Rf=0.44 in ethyl acetate/heptane eluent: 2/8

silica chromatography in ethyl acetate/heptane eluent: 1/9.

[α]_{D}²⁸=−66.8 (c=1.0; CHCl₃) for an enantiomeric excess of 98.7%.

HPLC: t_{R(min)}=6.8 min and t_{R(dom)}=7.5 min (Chiralpak IA, hexane/isopropanol: 95/5, 1 mL/min)

¹H NMR (CDCl₃, 300 MHz, δ): 1.24 (3H, d, ³J=6.3 Hz, H1), 1.25 (3H, d, ³J=6.0 Hz, H1′), 1.45 (9H, s, H7), 3.28-3.41 (2H, m, H8), 4.49-4.55 (1H, m, H4), 5.03 (1H, hept, ³J=6.3 Hz, H2), 5.14 (1H, d 1, ³J=7.8 Hz, NH), 6.81 (1H, d 1, ³J=3.0 Hz, H12), 6.93 (1H, dd, ³J=5.1 Hz, ⁴J=3.0 Hz, H11), 7.16 (1H, dd, ³J=5.1 Hz, ⁴J=0.9 Hz, H10).

¹³C NMR (CDCl₃, 75 MHz, δ): 21.8 (C1), 28.3 (C7), 32.4 (C8), 54.3 (C4), 69.4 (C2), 79.9 (C6), 124.7 (C12), 126.7 (C10 or C11), 126.8 (C10 or C11), 137.6 (C9), 155.0 (C5), 170.7 (C3).

HRMS: Calculated for C₁₅H₂₃O₄NNaS: 336.1240. Found: 336.1239.

### Example 27

### (R)-isopropyl 2-tert-butoxycarbonylamino-3-(furan-3-yl)propanoate

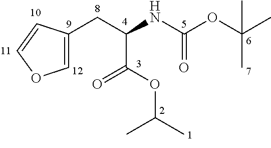 [SMILES: CC(C)OC(=O)[C@@H](Cc1ccoc1)NC(=O)OC(C)(C)C]

The following are introduced into a tubular reactor under an argon atmosphere: 6 mmol of 3-furaneboronic acid (692 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 104.7 mg of product is obtained (general procedure 1).
Colourless oil Yield=12% ee=99.9%

TLC: Rf=0.34 in ethyl acetate/heptane eluent: 2/8

silica chromatography in ethyl acetate/heptane eluent: 1/9.

[α]_{D}²⁷=−29.2 (c=1.0; CHCl₃) for an enantiomeric excess of 99.9%.

HPLC: t_{R(min)}=7.2 min and t_{R(dom)}=8.0 min (Chiralpak IA, hexane/isopropanol: 95/5, 1 mL/min)

¹H NMR (CDCl₃, 300 MHz, δ): 1.23 (3H, d, ³J=6.3 Hz, H1), 1.24 (3H, d, ³J=6.3 Hz, H1′), 1.44 (9H, s, H7), 2.86-2.99 (2H, m, H8), 4.45 (1H, q app, ³J=7.5 Hz, H4), 4.99-5.09 (2H, m, NH and H2), 6.24 (1H, d 1, ³J=0.9 Hz, H10), 7.23-7.25 (1H, m, H12), 7.35 (1H, t app, ³J=1.5 Hz, H11).

¹³C NMR (CDCl₃, 75 MHz, δ): 21.7 (C1), 21.8 (C1′), 27.7 (C8), 28.3 (C7), 53.5 (C4), 69.2 (C2), 79.8 (C6), 111.2 (C10), 119.1 (C9), 140.4 (C12), 143.0 (C11), 155.1 (C5), 171.3 (C3).

HRMS: Calculated for C₁₅H₂₃O₅NNa: 320.1468. Found: 320.1466.

### Example 28

### (R)-isopropyl 2-tert-butoxycarbonylamino-3-(furan-2-yl)propanoate

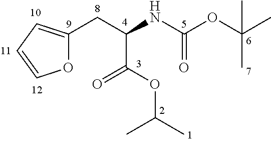 [SMILES: CC(C)OC(=O)[C@@H](Cc1ccco1)NC(=O)OC(C)(C)C]

The following are introduced into a tubular reactor under an argon atmosphere: 6 mmol of 2-furaneboronic acid (692 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 230.9 mg of product is obtained (general procedure 1).
Colourless oil Yield=26% ee=99.4%

TLC: Rf=0.39 in ethyl acetate/heptane eluent: 2/8

silica chromatography in ethyl acetate/heptane eluent: 1/9.

[α]_{D}²²=−32.4 (c=1.023; CHCl₃) for an enantiomeric excess of 99.4%.

HPLC: t_{R(min)}=6.9 min and t_{R(dom)}=7.6 min (Chiralpak IA, hexane/isopropanol: 95/5, 1 mL/min)

¹H NMR (CDCl₃, 300 MHz, δ): 1.17 (3H, d, ³J=6.3 Hz, H1), 1.18 (3H, d, ³J=6.3 Hz, H1′), 1.39 (9H, s, H7), 3.08-3.10 (2H, m, H8), 4.46 (1H, q 1 app, ³J=7.8 Hz, H4), 4.96 (1H, hept, ³J=6.3 Hz, H2), 5.16 (1H, Br d, ³J=8.1 Hz, NH), 6.03 (1H, d, ³J=3.3 Hz, H10), 6.21-6.23 (1H, m, H11), 7.26 (1H, d, ³J=0.9 Hz, H12).

¹³C NMR (CDCl₃, 75 MHz, δ): 21.6 (C1), 21.7 (C1′), 28.2 (C7), 30.9 (C8), 52.7 (C4), 69.1 (C2), 79.7 (C6), 107.8 (C10), 110.2 (C11), 141.9 (C12), 150.5 (C9), 155.1 (C5), 170.9 (C3).

HRMS: Calculated for C₁₅H₂₃O₅NNa: 320.1468. Found: 320.1465.

### Example 29

### (R)-isopropyl 2-tert-butoxycarbonylamino-3-phenylpropanoate

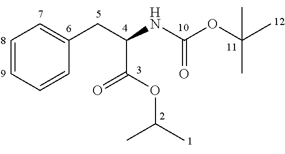 [SMILES: CC(C)OC(=O)[C@@H](Cc1ccccc1)NC(=O)OC(C)(C)C]

The following are introduced into a tubular reactor under an argon atmosphere: 1 mmol of phenylboronic acid (122 mg) and 0.5 mmol of isopropyl 2-tert-butoxycarbonylaminoacrylate (114.7 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 2 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 127.6 mg of product is obtained (general procedure 1).
Colourless oil Yield=83% ee=99.2%

TLC: Rf=0.52 in ethyl acetate/cyclohexane eluent: 2/8

silica chromatography in ethyl acetate/cyclohexane eluent: from 5/95 to 1/9.
[α]_{D}²¹=−36.3 (c=1; CHCl₃) for an enantiomeric excess of 99.2%.

HPLC: t_{R(min)}=7.4 min and t_{R(dom)}=8.4 min (Chiralpak AS-H, hexane/isopropanol: 95/5, 1 mL/min)

¹H NMR (CDCl₃, 300 MHz, δ): 1.18 (3H, d, ³J=6.3 Hz, H1), 1.21 (3H, d, ³J=6.3 Hz, H1′), 1.42 (9H, s, H12), 3.05-3.09 (2H, m, H5), 4.51 (1H, q app, ³J=7.8 Hz, H4), 4.96-5.02 (2H, H2 and NH), 7.13-7.31 (5H, m, H7, H8, H9).

¹³C NMR (CDCl₃, 75 MHz, δ): 21.7 (C1), 21.8 (C1′), 28.3 (C12), 38.4 (C5), 54.5 (C4), 69.1 (C2), 79.7 (C11), 126.9 (C9), 128.4 (C7), 129.4 (C8), 136.1 (C6), 155.1 (C10), 171.3 (C3).

### Example 30

### (R)-isopropyl 2-tert-butoxycarbonylamino-3-(2-methylphenyl)propanoate

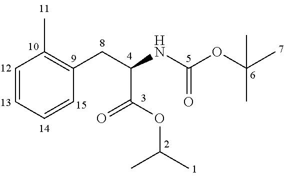 [SMILES: Cc1ccccc1C[C@@H](NC(=O)OC(C)(C)C)C(=O)OC(C)C]

The following are introduced into a tubular reactor under an argon atmosphere: 6 mmol of 2-methylphenylboronic acid (816 mg) and 3 mmol of isopropyl 2-tert-butoxycarbonylaminoacrylate (698 mg), 1.5 mol % of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 763.4 mg of product is obtained (general procedure 1).
White solid MP=72° C. Yield=79% ee=99.9%

TLC: Rf=0.59 in ethyl acetate/cyclohexane eluent: 2/8

silica chromatography in ethyl acetate/cyclohexane eluent: from 2/98 to 5/95 then 10/90.

[α]_{D}²³=−24.9 (c=1; CHCl₃) for an enantiomeric excess of 99.9%.

HPLC: t_{R(min)}=7.3 min and t_{R(dom)}=8.2 min (Chiralpak IA, hexane/isopropanol: 95/5, 1 mL/min)

¹H NMR (CDCl₃, 300 MHz, δ): 1.11 (3H, d, ³J=6.3 Hz, H1), 1.21 (3H, d, ³J=6.3 Hz, H1′), 1.40 (9H, s, H7), 2.35 (3H, s, H11), 2.97 (1H, dd, ²J=13.8 Hz, ³J=7.2 Hz, H8), 3.10 (1H, dd, ²J=13.8 Hz, ³J=6.6 Hz, H8′), 4.43 (1H, q app, ³J=7.5 Hz, H4), 4.93-5.04 (2H, m, H2 and NH), 7.05-7.16 (4H, m, H12, H13, H14, H15).

¹³C NMR (CDCl₃, 75 MHz, δ): 19.4 (C11), 21.5 (C1), 21.8 (C1′), 28.3 (C7), 36.3 (C8), 53.8 (C4), 69.0 (C2), 79.7 (C6), 125.9 (C13 or C14), 127.0 (C13 or C14), 130.0 (C12 or C15), 130.4 (C12 or C15), 134.6 (C10), 136.8 (C9), 155.0 (C5), 171.8 (C3).

### Example 31

### (R)-isopropyl 2-tert-butoxycarbonylamino-3-(4-methylphenyl)propanoate

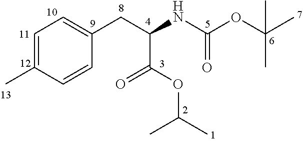 [SMILES: Cc1ccc(C[C@@H](NC(=O)OC(C)(C)C)C(=O)OC(C)C)cc1]

The following are introduced into a tubular reactor under an argon atmosphere: 6 mmol of 4-methylphenylboronic acid (816 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 827.4 mg of product is obtained (general procedure 1).
White solid MP=64° C. Yield=86% ee=98%

TLC: Rf=0.47 in ethyl acetate/cyclohexane eluent: 2/8

silica chromatography in ethyl acetate/cyclohexane eluent: 1/9.

[α]_{D}²⁸=−33.6 (c=1; CHCl₃) for an enantiomeric excess of 98%.

HPLC: t_{R(min)}=7.1 min and t_{R(dom)}=7.8 min (Chiralpak IA, hexane/isopropanol: 95/5, 1 mL/min)

¹H NMR (CDCl₃, 300 MHz, δ): 1.20 (3H, d, ³J=6.3 Hz, H1), 1.22 (3H, d, ³J=6.0 Hz, H1′), 1.42 (9H, s, H7), 2.31 (3H, s, H13), 2.97-3.09 (2H, m, H8), 4.49 (1H, q app, ³J=7.8 Hz, H4), 4.95-5.05 (2H, m, H2 and NH), 7.02 (2H, d, ³J=7.8 Hz, H10), 7.09 (2H, d, ³J=7.8 Hz, H11).

¹³C NMR (CDCl₃, 75 MHz, δ): 21.0 (C13), 21.7 (C1), 21.8 (C1′), 28.3 (C7), 37.8 (C8), 54.5 (C4), 69.0 (C2), 79.7 (C6), 129.1 and 129.3 (C10 and C11), 132.9 (C9), 136.5 (C12), 155.1 (C5), 171.4 (C3).

HRMS: Calculated for C₁₈H₂₇NO₄Na: 344.1823. Found: 344.1835.

### Example 32

### (R)-isopropyl 2-tert-butoxycarbonylamino-3-(3,5-dimethylphenyl)propanoate

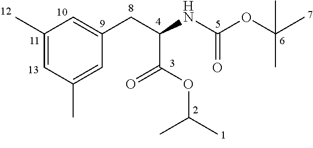 [SMILES: Cc1cc(C)cc(C[C@@H](NC(=O)OC(C)(C)C)C(=O)OC(C)C)c1]

The following are introduced into a tubular reactor under an argon atmosphere: 6 mmol of 3,5-dimethylphenylboronic acid (900 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 871 mg of product is obtained (general procedure 1).
Yellow oil Yield=87% ee=99.9%

TLC: Rf=0.62 in ethyl acetate/cyclohexane eluent: 2/8

silica chromatography in ethyl acetate/cyclohexane eluent: from 1/99 to 10/90.

[α]_{D}²²=−44.6 (c=1; CHCl₃) for an enantiomeric excess of 99.9%.

HPLC: t_{R(min)}=9.4 min and t_{R(dom)}=10.5 min (Chiralpak AD-H, hexane/isopropanol: 95/5, 1 mL/min)

¹H NMR (CDCl₃, 300 MHz, δ): 1.20 (3H, d, ³J=6.3 Hz, H1), 1.23 (3H, d, ³J=6.3 Hz, H1), 1.43 (9H, s, H7), 2.27 (6H, s, H12), 2.98-3.02 (2H, m, H8), 4.47 (1H, q 1 app, ³J=7.8 Hz, H4), 4.95-5.05 (2H, m, H2 and NH), 6.75 (2H, s, H10), 6.87 (1H, s, H13).

¹³C NMR (CDCl₃, 75 MHz, δ): 21.2 (C12), 21.7 (C1), 21.8 (C1′), 28.3 (C7), 38.0 (C8), 54.5 (C4), 69.0 (C2), 79.7 (C6), 127.2 (C10), 128.5 (C13), 135.9 (C9), 137.8 (C9) 155.1 (C5), 171.5 (C3). HRMS: Calculated for C₁₉H₂₉NO₄Na: 358.1989. Found: 358.1991.

### Example 33

### (R)-isopropyl 2-tert-butoxycarbonylamino-3-(indol-5-yl)propanoate

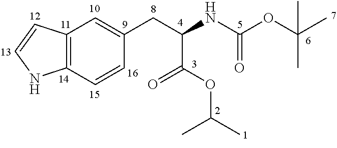 [SMILES: CC(C)OC(=O)[C@@H](Cc1ccc2[nH]ccc2c1)NC(=O)OC(C)(C)C]

The following are introduced into a tubular reactor under an argon atmosphere: 6 mmol of 5-indolylboronic acid (860 mg) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 518.1 mg of product is obtained (general procedure 1).
White solid MP=110° C. Yield=50% ee=98%

TLC: Rf=0.15 in ethyl acetate/cyclohexane eluent: 2/8

silica chromatography in ethyl acetate/cyclohexane eluent: from 15/85 to 2/8.

[α]_{D}²⁵=−42.4 (c=1; CHCl₃) for an enantiomeric excess of 98%.

HPLC: t_{R(min)}=12.0 min and t_{R(dom)}=16.2 min (Chiralpak AD-H, hexane/isopropanol: 9/1, 1 mL/min)

¹H NMR (CD₃OD, 300 MHz, δ): 1.11 (3H, d, ³J=6.3 Hz, H1), 1.22 (3H, d, ³J=6.3 Hz, H1′), 1.40 (9H, s, H7), 3.01 (1H, dd, ²J=13.6 Hz, ³J=8.1 Hz, H8), 3.13 (1H, dd, ²J=13.6 Hz, ³J=6.3 Hz, H8′), 4.30-4.35 (1H, m, H4), 4.96 (1H, hept, ³J=6.3 Hz, H2), 6.39 (1H, dd, ³J=3.0 Hz, ⁴J=0.9 Hz, H12), 6.97 (1H, dd, ³J=8.2 Hz, J=1.5 Hz, H16), 7.21 (1H, d, ³J=3.0 Hz, H13), 7.31 (1H, d, ³J=8.2 Hz, H15), 7.39 (1H, s, H10).

¹³C NMR (CD₃OD, 75 MHz, δ): 21.9 (C1), 22.0 (C1′), 28.7 (C7), 39.1 (C8), 57.5 (C4), 69.9 (C2), 80.5 (C6), 102.1 (C12), 112.1 (C15), 121.8 (C10), 123.6 (C13), 125.9 (C16), 128.2 (C11), 129.6 (C9), 136.8 (C14), 157.8 (C5), 173.8 (C3).

HRMS: Calculated for C₁₉H₂₆O₄N₂Na: 369.1785. Found: 369.1787.

### Example 34

### (R)-isopropyl 2-tert-butoxycarbonylamino-3-(benzothien-2-yl)propanoate

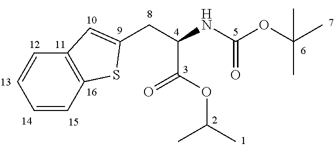 [SMILES: CC(C)OC(=O)[C@@H](Cc1cc2ccccc2s1)NC(=O)OC(C)(C)C]

The following are introduced into a tubular reactor under an argon atmosphere: 6 mmol of 2-benzothienylboronic acid (1.068 g) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 692.9 mg of product is obtained (general procedure 1).
Beige solid MP=96° C. Yield=64% ee=99%

TLC: Rf=0.46 in ethyl acetate/cyclohexane eluent: 2/8

silica chromatography in ethyl acetate/cyclohexane eluent: from 5/95 to 1/9.

[α]_{D}²⁸=−54.4 (c=1; CHCl₃) for an enantiomeric excess of 99%.

HPLC: t_{R(min)}=18.8 min and t_{R(dom)}=21.1 min (Chiralpak AD-H, hexane/isopropanol: 9/1, 0.5 mL/min)

¹H NMR (CDCl₃, 300 MHz, δ): 1.25 (3H, d, ³J=6.3 Hz, H1), 1.26 (3H, d, ³J=6.3 Hz, H1′), 1.46 (9H, s, H7), 3.42-3.45 (2H, m, H8), 4.58-4.64 (1H, m, H4), 5.06 (1H, hept, ³J=6.3 Hz, H2), 5.23 (1H, d 1, ³J=7.8 Hz, NH), 7.04 (1H, s, H10), 7.25-7.35 (2H, m, H13, H14), 7.67-7.70 (1H, m, H12), 7.75-7.78 (1H, m, H15).

¹³C NMR (CDCl₃, 75 MHz, δ): 21.8 (C1), 28.3 (C7), 33.3 (C8), 54.0 (C4), 69.6 (C2), 80.0 (C6), 122.1 (C10), 123.0, 123.5, 123.9, 124.2, 138.9, 139.7, 139.9, 155.1 (C5), 170.6 (C3).

HRMS: Calculated for C₁₉H₂₅O₄NNaS: 386.1397. Found: 386.1400.

### Example 35

### (R)-isopropyl 2-tert-butoxycarbonylamino-3-(6-methoxynaphth-2-yl)propanoate

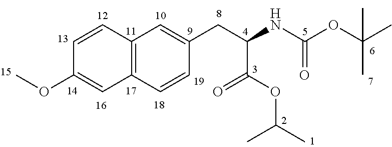 [SMILES: COc1ccc2cc(C[C@@H](NC(=O)OC(C)(C)C)C(=O)OC(C)C)ccc2c1]

The following are introduced into a tubular reactor under an argon atmosphere: 3 mmol of 6-methoxynaphth-2-ylboronic acid (666 mg) and 1.5 mmol of isopropyl 2-tert-butoxycarbonylaminoacrylate (353 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 6 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 450.3 mg of product is obtained (general procedure 1).
White solid MP=95° C. Yield=77% ee=98.5%

TLC: Rf=0.39 in ethyl acetate/cyclohexane eluent: 2/8

silica chromatography in ethyl acetate/cyclohexane eluent: 15/85.
[α]_{D}²⁸=−36.3 (c=1; CHCl₃) for an enantiomeric excess of 98.5%.

HPLC: t_{R(min)}=8.5 min and t_{R(dom)}=10.7 min (Chiralpak AS-H, hexane/isopropanol: 9/1, 1 mL/min)

¹H NMR (CDCl₃, 300 MHz, δ): 1.17 (3H, d, ³J=6.3 Hz, H1), 1.21 (3H, d, ³J=6.3 Hz, H1′), 1.41 (9H, s, H7), 3.15-3.27 (2H, m, H8), 3.91 (3H, s, H15), 4.58 (1H, q 1 app, ³J=7.5 Hz, H4), 4.97-5.06 (2H, m, H2 and NH), 7.10-7.17 (2H, m, H13, H16), 7.26 (1H, dd, ³J=9.0 Hz, ⁴J=1.8 Hz, H19), 7.52 (1H, s 1, H10), 7.66 (1H, d, ³J=8.7 Hz, H12 or H18), 7.68 (1H, d, ³J=8.4 Hz, H12 or H18).

¹³C NMR (CDCl₃, 75 MHz, δ): 21.7 (C1), 21.8 (C1′), 28.3 (C7), 38.3 (C8), 54.6 (C4), 55.3 (C15), 69.1 (C2), 79.8 (C6), 105.6 (C16), 118.9 (C13), 126.9, 127.97, 128.03, 128.9, 129.0, 131.3, 133.5, 155.1 (C5), 157.5 (C14), 171.4 (C3).

HRMS: Calculated for C₂₂H₂₉NO₅Na: 410.1938. Found: 410.1942.

### Example 36

### (R)-isopropyl 2-tert-butoxycarbonylamino-3-(naphth-1-yl)propanoate

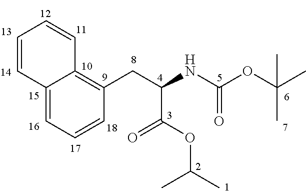 [SMILES: CC(C)OC(=O)[C@@H](Cc1cccc2ccccc12)NC(=O)OC(C)(C)C]

The following are introduced into a tubular reactor under an argon atmosphere: 6 mmol of naphth-1-ylboronic acid (1.032 g) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 248.7 mg of product is obtained (general procedure 1).
Yellow oil Yield=23% ee=98.5%

TLC: Rf=0.38 in ethyl acetate/cyclohexane eluent: 2/8

silica chromatography in ethyl acetate/cyclohexane eluent: from 5/95 to 15/85.

[α]_{D}²⁸=−15.3 (c=1; CHCl₃) for an enantiomeric excess of 98.5%.

HPLC: t_{R(min)}=11.8 min and t_{R(dom)}=14.5 min (Chiralpak AD-H, hexane/isopropanol: 9/1, 1 mL/min)

¹H NMR (CDCl₃, 300 MHz, δ): 1.02 (3H, d, ³J=6.3 Hz, H1), 1.14 (3H, d, ³J=6.3 Hz, H1′), 1.42 (9H, s, H7), 3.49 (1H, dd, ²J=14.1 Hz, ³J=6.9 Hz, H8), 3.57 (1H, dd, ²J=14.1 Hz, ³J=6.9 Hz, H8′), 4.68 (1H, q app, ³J=7.5 Hz, H4), 4.92 (1H, hept, ³J=6.3 Hz, H2), 5.12 (1H, d 1, ³J=7.8 Hz, NH), 7.26-7.31 (1H, m, H18), 7.38 (1H, t, ³J=8.1 Hz, H17), 7.48-7.55 (2H, m, H12, H13), 7.76 (1H, d, ³J=8.1 Hz, H16), 7.85 (1H, d, ³J=7.8 Hz, H14), 8.13 (1H, d, ³J=8.1 Hz, H11).

¹³C NMR (CDCl₃, 75 MHz, δ): 21.5 (C1), 21.7 (C1′), 28.3 (C7), 35.8 (C8), 54.6 (C4), 69.1 (C2), 79.7 (C6), 123.8, 125.3, 125.7, 126.2, 127.6, 127.8, 128.7, 132.3, 132.7, 133.9, 155.0 (C5), 171.7 (C3).

HRMS: Calculated for C₂₁H₂₇O₄NNa: 380.1832. Found: 380.1836.

### Example 37

### (R)-isopropyl 2-tert-butoxycarbonylamino-3-(naphth-2-yl)propanoate

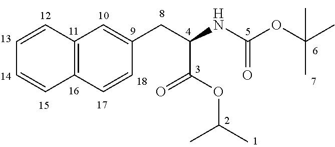 [SMILES: CC(C)OC(=O)[C@@H](Cc1ccc2ccccc2c1)NC(=O)OC(C)(C)C]

The following are introduced into a tubular reactor under an argon atmosphere: 6 mmol of naphth-2-ylboronic acid (1.032 g) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 0.997 g of product is obtained (general procedure 1).
White solid MP=113° C. Yield=93% ee=98.9%

TLC: Rf=0.5 in ethyl acetate/cyclohexane eluent: 2/8

silica chromatography in ethyl acetate/cyclohexane eluent: from 2/98 to 90/10.

[α]_{D}²⁸=−37.1 (c=1; CHCl₃) for an enantiomeric excess of 98.9%.

HPLC: t_{R(min)}=34.3 min and t_{R(dom)}=41.3 min (Chiralpak AD-H, hexane/isopropanol: 98/2, 1 mL/min)

¹H NMR (CDCl₃, 300 MHz, δ): 1.17 (3H, d, ³J=6.3 Hz, H1), 1.21 (3H, d, ³J=6.0 Hz, H1′), 1.41 (9H, s, H7), 3.14-3.32 (2H, m, H8), 4.60 (1H, q app, ³J=7.5 Hz, H4), 4.98-5.06 (2H, m, H2 and NH), 7.30 (1H, dd, ³J=8.5 Hz, ⁴J=1.7 Hz, H18), 7.42-7.49 (2H, m, H13, H14), 7.60 (1H, s 1, H10), 7.75-7.82 (3H, m, H17, H15, H12).

¹³C NMR (CDCl₃, 75 MHz, δ): 21.7 (C1), 21.8 (C1′), 28.3 (C7), 38.5 (C8), 54.6 (C4), 69.2 (C2), 79.8 (C6), 125.6, 126.1, 127.5, 127.6, 128.2, 132.4, 133.4, 133.7, 155.1 (C5), 171.4 (C3).

### Example 38

### (R)-isopropyl 2-tert-butoxycarbonylamino-3-(dibenzofuran4-yl)propanoate

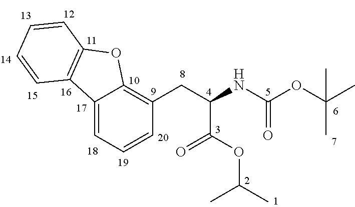 [SMILES: CC(C)OC(=O)[C@@H](Cc1cccc2c1oc1ccccc12)NC(=O)OC(C)(C)C]

The following are introduced into a tubular reactor under an argon atmosphere: 6 mmol of 4-dibenzofuraneboronic acid (1.272 g) and 3 mmol of isopropyl 2-tert-butoxycarbonylaminoacrylate (698 mg), 1.5 mol % of dimer of chlorobis(ethylene)rhodium(I) [RhCl(═)₂]₂, 3.3 mol % of (S)-Difluorphos and one equivalent of NaHCO₃. The mixture is stirred under vacuum for about ten minutes and is then placed under argon. 12 mL of isopropanol is then introduced. After a succession of two vacuum/argon cycles, the reactor is immersed in a bath preheated to 25° C. After stirring for 20 hours, the mixture is concentrated under vacuum. The addition product is then purified by silica gel chromatography. 1.035 g of product is obtained (general procedure 1).
Beige solid MP=116° C. Yield=87% ee=99.5%

TLC: Rf=0.47 in ethyl acetate/cyclohexane eluent: 2/8

silica chromatography in ethyl acetate/cyclohexane eluent: 1/9.

[α]_{D}²⁰=−42.1 (c=1; CHCl₃) for an enantiomeric excess of 99.5%.

HPLC: t_{R(min)}=17.7 min and t_{R(dom)}=20.1 min (Chiralpak AD-H, hexane/isopropanol: 95/5, 1 mL/min)

¹H NMR (CDCl₃, 300 MHz, δ): 1.15 (3H, d, ³J=6.3 Hz, H1), 1.18 (3H, d, ³J=6.3 Hz, H1′), 1.37 (9H, s, H7), 3.44-3.46 (2H, m, H8), 4.73 (1H, q app, ³J=6.3 Hz, H4), 4.98 (1H, hept, ³J=6.3 Hz, H2), 5.18 (1H, d 1, ³J=8.1 Hz, NH), 7.26-7.28 (2H, m, H19, H₂O), 7.36 (1H, td, ³J=7.9 Hz, J=1.5 Hz, H14), 7.46 (1H, td, ³J=8.4 Hz, J=1.5 Hz, H13), 7.57 (1H, d, ³J=9.0 Hz, H12), 7.84-7.86 (1H, m, H18), 7.94 (1H, dd, ³J=7.5 Hz, J=0.6 Hz, H15).

¹³C NMR (CDCl₃, 75 MHz, δ): 21.6 (C1), 21.7 (C1′), 28.2 (C7), 32.7 (C8), 54.0 (C4), 69.2 (C2), 79.6 (C6), 111.7 (C19), 119.4, 120.4, 120.7, 122.7, 122.8, 124.0, 124.4, 127.1, 128.5, 155.1 (C5), 156.0 (C10), 171.4 (C3).

HRMS: Calculated for C₂₃H₂₇NO₅Na: 420.1781. Found: 420.1788.

### Example 39

### (R)-isopropyl 2-((tert-butoxycarbonyl)amino)-3-(3,4-dimethoxyphenyl)propanoate

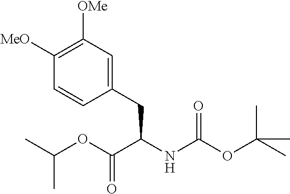 [SMILES: COc1ccc(C[C@@H](NC(=O)OC(C)(C)C)C(=O)OC(C)C)cc1OC]

Obtained following general procedure 1.
Yellow solid Yield=54% ee=99.4%

TLC: Rf=0.15 in heptane/EtOAc eluent 80:20

silica chromatography, with gradient of heptane/EtOAc eluent 100:0 to 85:15.

HPLC: t₁=6.70 min (R), t₂=7.57 min (S) (Chiralpak IB, hexane/isopropanol: 95/5).

¹H NMR (CDCl₃, 300 MHz, δ): 6.76 (d, 1H, J=7.8 Hz), 6.63-6.67 (m, 2H), 5.03-4.94 (2H, m), 4.46 (q, 1H, J=6 Hz), 3.83 (s, 6H), 3.00 (t, 2H, J=5.4 Hz), 1.40 (s, 9H), 1.21 (d, 3H, J=6 Hz), 1.19 (d, 3H, J=6 Hz).

¹³C NMR (CDCl₃, 75 MHz, δ): 21.84, 21.89, 37.9, 54.6, 55.8, 55.9, 69.1, 79.8, 111.2, 112.5, 121.6, 128.6, 148.1, 148.8, 155.1, 171.5.

### Example 40

### (R)-isopropyl 2-((tert-butoxycarbonyl)amino)-3-(3,4-dichlorophenyl)propanoate

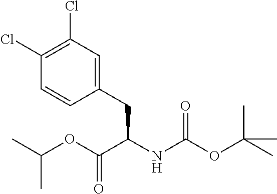 [SMILES: CC(C)OC(=O)[C@@H](Cc1ccc(Cl)c(Cl)c1)NC(=O)OC(C)(C)C]

Obtained following general procedure 1.
Yellow oil Yield=64% ee=97%

TLC: Rf=0.34 in heptane/EtOAc eluent 75:25

silica chromatography, with gradient of heptane/EtOAc eluent 100:0 to 85:15

HPLC: t₁=4.92 min (R), t₂=5.60 min (S) (Chiralpak IC, hexane/ethanol: 95/5).

¹H NMR (CDCl₃, 300 MHz, δ): 7.31 (d, 1H, J=8.1 Hz), 7.21-7.20 (m, 1H), 6.96 (dd, 1H, J=2.1 Hz, 8.4 Hz), 5.08-4.94 (2H, m), 4.44 (q, 1H, J=6 Hz), 3.09-2.91 (m, 2H), 1.39 (s, 9H), 1.20 (d, 3H, J=6 Hz), 1.18 (d, 3H, J=6 Hz).

¹³C NMR (CDCl₃, 75 MHz, δ): 21.74, 21.79, 37.4, 54.3, 69.5, 80.0, 128.8, 130.3, 131.0, 131.4, 132.2, 136.6, 155.0, 170.8.

### Example 41

### (R)-isopropyl 3-([1,1′-biphenyl]-4-yl)-2-((tert-butoxycarbonyl)amino)propanoate

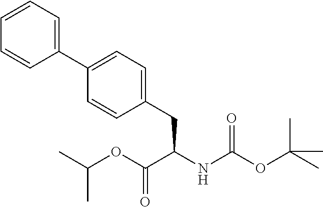 [SMILES: CC(C)OC(=O)[C@@H](Cc1ccc(-c2ccccc2)cc1)NC(=O)OC(C)(C)C]

Obtained following general procedure 1, with reaction temperature of 40° C.
White solid Yield=78% ee=97.4%

TLC: Rf=0.33 in heptane/EtOAc eluent 75:25

silica chromatography, with gradient of heptane/EtOAc eluent 100:0 to 85:15.

HPLC: t₁=6.37 min (R), t₂=7.19 min (S) (Chiralpak IC, hexane/ethanol: 95/5).

¹H NMR (CDCl₃, 300 MHz, δ): 7.59-7.21 (m, 9H), 5.07-4.99 (m, 2H), 4.56 (q, 1H, J=6.1 Hz), 3.12 (t, 2H, J=5.7 Hz), 1.43 (s, 9H), 1.24 (d, 3H, J=6.3 Hz), 1.21 (d, 3H, J=6.3 Hz).

¹³C NMR (CDCl₃, 75 MHz, δ): 21.7, 21.8, 38.0, 54.5, 69.2, 79.8, 127.0, 127.20, 127.28, 128.8, 129.9, 135.2, 139.8, 140.8, 155.1, 171.4.

### Example 42

### (R)-isopropyl 2-((tert-butoxycarbonyl)amino)-3-(4-(tert-butyl)phenyl)propanoate

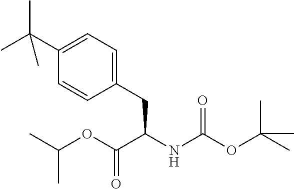 [SMILES: CC(C)OC(=O)[C@@H](Cc1ccc(C(C)(C)C)cc1)NC(=O)OC(C)(C)C]

Obtained following general procedure 1, with reaction temperature of 40° C.
Yellow oil Yield=71% ee=96%

TLC: Rf=0.49 in heptane/EtOAc eluent 75:25

silica chromatography, with gradient of heptane/EtOAc eluent 100:0 to 85:15.

HPLC: t₁=5.16 min (R), t₂=5.82 min (S) (Chiralpak IC, hexane/ethanol: 95/5).

¹H NMR (CDCl₃, 300 MHz, δ): 7.29 (d, 2H, J=8.4 Hz), 7.07 (d, 2H, J=8.1 Hz), 5.03-4.95 (m, 2H), 4.50 (q, 1H, J=6.3 Hz), 3.03 (d, 2H, J=6 Hz), 1.41 (s, 9H), 1.29 (s, 9H), 1.20 (d, 3H, J=6.3 Hz), 1.17 (d, 3H, J=6.3 Hz).

¹³C NMR (CDCl₃, 75 MHz, δ): 21.7, 21.8, 28.4, 29.7, 31.4, 38.3, 54.6, 69.0, 79.8, 125.4, 129.2, 133, 149.8, 155.1, 171.5.

### Example 43

### (R)-isopropyl 2-((tert-butoxycarbonyl)amino)-3-(3-(tert-butyldimethylsilyl)phenyl)propanoate

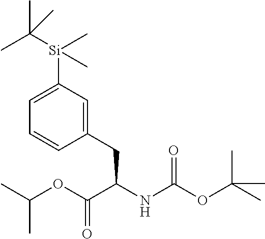 [SMILES: CC(C)OC(=O)[C@@H](Cc1cccc([Si](C)(C)C(C)(C)C)c1)NC(=O)OC(C)(C)C]

Obtained following general procedure 1, with reaction temperature of 40° C.
Yellow oil Yield=95% ee=98%

TLC: Rf=0.37 in heptane/EtOAc eluent 75:25

silica chromatography, with gradient of heptane/EtOAc eluent 100:0 to 85:15.

HPLC: t₁=6.00 min (R), t₂=6.27 min (S) (Chiralpak IA, hexane/isopropanol: 98/2).

¹H NMR (CDCl₃, 300 MHz, δ): 7.11 (t, 1H, J=7.8 Hz), 6.72-6.68 (m, 2H), 6.62 (s, 1H), 5.03-4.95 (m, 2H), 4.48 (q, 1H, J=5.7 Hz), 3.01 (d, 2H, J=5.7 Hz), 1.41 (s, 9H), 1.20 (d, 3H, J=6 Hz), 1.19 (d, 3H, J=6 Hz), 0.96 (s, 9H), 0.17 (s, 6H).

¹³C NMR (CDCl₃, 75 MHz, δ): −4.34, −4.33, 17.9, 21.80, 21.87, 25.7, 28.4, 38.1, 54.4, 69.1, 79.7, 118.6, 121.2, 122.5, 129.4, 137.9, 155.18, 155.63, 171.4.

### Example 44

### (R)-isopropyl 2-((tert-butoxycarbonyl)amino)-3-(4-(methylsulphonyl)phenyl)propanoate

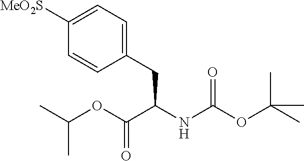 [SMILES: CC(C)OC(=O)[C@@H](Cc1ccc(S(C)(=O)=O)cc1)NC(=O)OC(C)(C)C]

Obtained following general procedure 1, with reaction temperature of 40° C.
White solid Yield=47% ee=96%

TLC: Rf=0.52 in heptane/EtOAc eluent 50:50

silica chromatography, with gradient of heptane/EtOAc eluent 100:0 to 85:15.

HPLC: t₁=11.93 min (R), t₂=14.84 min (S) (Chiralpak IB, hexane/ethanol: 95/5).

¹H NMR (CDCl₃, 300 MHz, δ): 7.83 (d, 2H, J=8.4 Hz), 7.34 (d, 2H, J=8.1 Hz), 5.09 (d, 1H, J=7.8 Hz), 4.99 (sext., 1H, J=6.3 Hz), 4.52 (q, 1H, J=6.3 Hz), 3.24-3.07 (m, 2H), 3.00 (s, 3H), 1.38 (s, 9H), 1.20 (d, 3H, J=6.9 Hz), 1.18 (d, 3H, J=6.9 Hz).

¹³C NMR (CDCl₃, 75 MHz, δ): 21.7, 21.8, 28.3, 38.4, 44.5, 54.2, 69.7, 80.3, 116.1, 127.5, 129.7, 130.5, 139.1, 143.0, 155.1, 170.8.

### Example 45

### (R)-isopropyl 2-((tert-butoxycarbonyl)amino)-3-(4-isopropylphenyl)propanoate

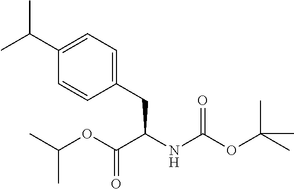 [SMILES: CC(C)OC(=O)[C@@H](Cc1ccc(C(C)C)cc1)NC(=O)OC(C)(C)C]

Obtained following general procedure 1, with reaction temperature of 40° C.
Yellow oil Yield=50%

TLC: Rf=0.55 in heptane/EtOAc eluent 75:25

silica chromatography, with gradient of heptane/EtOAc eluent 100:0 to 85:15.

HPLC: t₁=4.49 min (R), t₂=4.92 min (S) (Chiralpak IC, hexane/ethanol: 90/10).

¹H NMR (CDCl₃, 300 MHz, δ): 7.13 (d, 2H, J=8.1 Hz), 7.05 (d, 2H, J=8.4 Hz), 5.03-4.94 (m, 2H), 4.49 (q, 1H, J=5.7 Hz), 3.02 (d, 2H, J=6 Hz), 2.86 (sext., 1H, J=6.9 Hz), 1.4 (s, 9H), 1.19 (m, 12H).

¹³C NMR (CDCl₃, 75 MHz, δ): 21.7, 21.8, 24.10, 24.13, 28.4, 33.8, 38.1, 54.6, 69.1, 79.8, 126.6, 129.4, 133.4, 147.5, 155.2, 171.5.

### Example 46

### (R)-2-tert-Butoxycarbonylamino-3-(3-phenoxy-phenyl)-propionic acid isopropyl ester

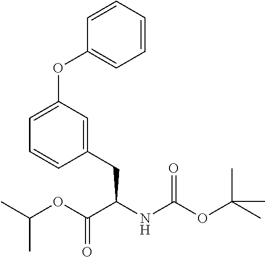 [SMILES: CC(C)OC(=O)[C@@H](Cc1cccc(Oc2ccccc2)c1)NC(=O)OC(C)(C)C]

Obtained following general procedure 1, with reaction temperature of 40° C.
Yellow oil Yield=69% ee=98%

TLC: Rf=0.40 in heptane/EtOAc eluent 75:25

silica chromatography, with gradient of heptane/EtOAc eluent 95:5 to 90:10.

HPLC: t₁=5.58 min (R), t₂=6.06 min (S) (Chiralpak IC, hexane/ethanol: 95/5).

¹H NMR (CDCl₃, 300 MHz, δ): 7.36-6.81 (m, 9H), 5.15-4.93 (m, 2H), 4.50 (q, 1H, J=5.7 Hz), 3.13-2.99 (m, 2H), 1.42 (s, 9H), 1.21 (d, 3H, J=5.7 Hz), 1.14 (d, 3H, J=5.7 Hz).

¹³C NMR (CDCl₃, 75 MHz, δ): 21.6, 28.3, 38.2, 54.4, 69.2, 80.0, 117.4, 118.9, 119.8, 123.3, 124.3, 129.8, 138.8, 155.3, 157.5, 171.2.

### Example 47

### (R)-isopropyl 2-tert-butoxycarbonylamino-3-(6-(piperidin-1-yl)pyridin-3-yl)propanoate

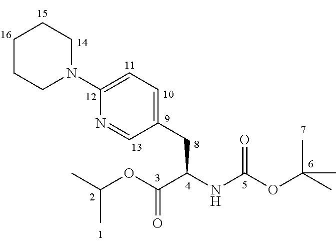 [SMILES: CC(C)OC(=O)[C@@H](Cc1ccc(N2CCCCC2)nc1)NC(=O)OC(C)(C)C]

Obtained from 2-(1-piperidyl)-5-(4,4,5,5-tetramethyl-1,3,2-dioxaborolan-2-yl)pyridine and isopropyl 2-tert-butoxycarbonylaminoacrylate (general procedure 2).
Brown oil Yield=70% ee=97.4%

TLC: Rf=0.24 in ethyl acetate/cyclohexane eluent: 2/8

silica chromatography in ethyl acetate/cyclohexane eluent: 2/8.

[α]_{D}²³=−35.4 (c=1; CHCl₃) for an enantiomeric excess of 97.4%.

HPLC: t_{R(min)}=13.8 min and t_{R(dom)}=16.4 min (Chiralpak AD-H, hexane/isopropanol: 9/1, 1 mL/min)

¹H NMR (CDCl₃, 300 MHz, δ): 1.21 (6H, d, ³J=6.3 Hz, H1), 1.40 (9H, s, H7), 1.61 (5H, s, H15, H16), 2.88 (1H, dd, ²J=14.0 Hz, ³J=5.7 Hz, H8), 2.96 (1H, dd, ²J=14.0 Hz, ³J=5.7 Hz, H8′), 4.43 (1H, q app, ³J=7.5 Hz, H4), 4.96-5.04 (2H, m, H2, NH), 6.57 (1H, d, ³J=8.7 Hz, H11), 7.23 (1H, dd, ³J=8.7 Hz, ⁴J=2.4 Hz, H10), 7.91 (1H, d, ⁴J=2.4 Hz, H13).

¹³C NMR (CDCl₃, 75 MHz, δ): 21.8 (C1), 24.7 (C16), 25.5 (C15), 28.3 (C7), 34.6 (C8), 46.4 (C14), 54.4 (C4), 69.1 (C2), 79.8 (C6), 106.9 (C11), 119.5 (C9), 138.4 (C10), 148.3 (C13), 155.1 (C12), 158.9 (C5), 171.2 (C3).

HRMS: Calculated for C₂₁H₃₃N₃O₄: 392.2544. Found: 392.2541.

1. A method for preparing chiral derivatives consisting of α- or β-amino acids or derivatives thereof with an enantiomeric excess of at least 95%, comprising steps of:
reacting a starting product consisting of an α-aminoacrylate with an organoboron derivative, with the aid of an electron-poor biphosphorus ligand in the presence of a catalyst containing a transition metal, at a temperature in the range from −20° C. to 70° C.;
wherein the reacting step is performed using a solvent and a proton donor element, the pKa of which in water is above 7, in the presence of a base belonging to an acid/base pair the pKa of which in water is above 4.

2. The method according to claim 1, in which the proton donor is the solvent and is selected from primary, secondary or tertiary alcohols containing from 1 to 8 carbon atoms selected from methanol, ethanol, n-propanol, n-butanol, isopropanol, sec-butanol, isobutanol and tert-butanol.

3. The method according to claim 1, in which the organoboron derivative has the following formula: 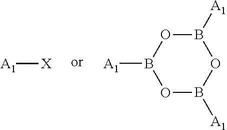
in which:
A₁ is selected from:
1. Aryls having rings with 6 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals with 1 to 15 carbon atoms,
with optionally substituted alkenyl radicals with 1 to 15 carbon atoms,
with optionally substituted alkynyl radicals with 1 to 15 carbon atoms,
with optionally substituted aryls with 6 to 12 carbon atoms,
with optionally substituted aromatic or non-aromatic heterocycles with 2 to 12 carbon atoms,
in which R^{a} and R^{b}, which may be identical or different, represent linear or branched, aromatic or heterocyclic non-aromatic or aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted linear or branched alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted aryls with 6 to 12 carbon atoms,
with optionally substituted aromatic or non-aromatic heterocycles having from 2 to 12 carbon atoms,
2. A₁ is further selected from the heterocycles or the heteroaryls having rings with 2 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
3. A₁ is further selected from linear or branched alkenyls having from 1 to 12 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
4. A₁ is further selected from the linear or branched alkynyls having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
5. A₁ is further selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
X is selected from B(OH)₂, B(OR)₂, BF₃M, B(OR′)₃M,
in which:
R is an alkyl group having from 1 to 14 carbon atoms, (OR)₂ optionally forming a ring between the two oxygen atoms. The groups (OR)₂ in particular originate from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and esters thereof,
or (OR)₂ in particular originates from diacids such as 2,2′-(methylazanediyl)diacetic acid (mida),
R′ is an alkyl group having from 1 to 14 carbon atoms, (OR)₃ optionally forming a ring between two of the oxygen atoms or a dicyclic ring between the three oxygen atoms. The groups (OR)₃ in particular originate from triols such as 2-(hydroxymethyl)-2-methylpropane-1,3-diol,
M represents the lithium ion Li⁺, sodium ion Na⁺, potassium ion K⁺, caesium ion Cs₊, ammonium ion R^{c}R^{d}R^{e}R^{f}N⁺ where R^{c}, R^{d}R^{e}, R^{f} are selected from H or a saturated carbon chain in particular having 1 to 6 carbon atoms selected independently of one another,
and in particular A₁-X represents A₁-B(OH)₂, A₁-B(OR)₂ or A₁-BF₃K,
in which A₁ has the same meaning as above.

4. The method according to claim 1, in which the starting product is a compound of formula: 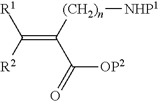
in which:
n is equal to 0 or 1,
R¹ and R² are selected, independently of one another, from alkyl or aromatic groups containing from 1 to 10 carbon atoms, provided that at least R¹ or R² is a hydrogen,
P¹ is an amine protective group selected from:
COR³, in which R³ represents a linear or branched alkyl, alkenyl, alkynyl group; benzyl, phthalimido (in this case NH is replaced with N) optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
COOR⁴, in which R⁴ represents an alkyl group, more particularly methyl, ethyl, propyl, benzyl, tert-butyl, but also alkenyl, alkynyl, linear or branched, benzyl optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P¹ is in particular selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine,
P² is a carboxylic acid protective group in particular selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkenyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkynyl groups having from 1 to 15 carbon atoms and optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched benzyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched silyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms.
P² is in particular selected from linear or branched alkyl groups having from 1 to 15 carbon atoms, optionally substituted, and in particular methyl, ethyl, isopropyl, tert-butyl and benzyl.

5. The method according to claim 1, in which the chiral α- and β-amino acids or derivatives thereof have the formula: 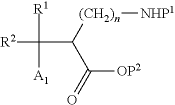
in which:
A₁ is selected from:
1. Aryls having rings with 6 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals with 1 to 15 carbon atoms,
with optionally substituted alkenyl radicals with 1 to 15 carbon atoms,
with optionally substituted alkynyl radicals with 1 to 15 carbon atoms,
with optionally substituted aryls with 6 to 12 carbon atoms,
with optionally substituted aromatic or non-aromatic heterocycles with 2 to 12 carbon atoms,
in which R^{a} and R^{b}, which may be identical or different, represent linear or branched, aromatic or heterocyclic non-aromatic or aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted linear or branched alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted aryls with 6 to 12 carbon atoms,
with optionally substituted aromatic or non-aromatic heterocycles having from 2 to 12 carbon atoms,
2. A₁ is further selected from the heterocycles or the heteroaryls having rings with 2 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
3. A₁ is further selected from linear or branched alkenyls having from 1 to 12 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃—NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
4. A₁ is further selected from the linear or branched alkynyls having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
5. A₁ is further selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P¹ is an amine protective group selected from:
COR³, in which R³ represents a linear or branched alkyl, alkenyl, alkynyl group; benzyl, phthalimido (in this case NH is replaced with N) optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
COOR⁴, in which R⁴ represents an alkyl group, more particularly methyl, ethyl, propyl, benzyl, tert-butyl, but also alkenyl, alkynyl, linear or branched, benzyl optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P¹ is in particular selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine,
P² is a carboxylic acid protective group in particular selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkenyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkynyl groups having from 1 to 15 carbon atoms and optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched benzyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched silyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is in particular selected from linear or branched alkyl groups having from 1 to 15 carbon atoms, optionally substituted, and in particular methyl, ethyl, isopropyl, tert-butyl and benzyl,
n is equal to 0 or 1,
R¹ and R² are selected, independently of one another, from alkyl or aromatic groups containing from 1 to 10 carbon atoms, provided that at least R¹ or R² is a hydrogen.

6. Process for preparing chiral α- or β-amino acids or derivatives thereof with an enantiomeric excess of at least 95%, comprising a step of reacting a starting product consisting of an α-aminoacrylate or of an α-aminomethyl acrylate with an organoboron derivative, with the aid of a solvent and a proton donor element, the pKa of which in water is above 7, in the presence of a base belonging to an acid/base pair with pKa in water above 4, of an electron-poor biphosphorus ligand, and of a catalyst containing a transition metal, at a temperature in the range from −20° C. to 70° C., making it possible to obtain protected chiral α- or β-amino acids or derivatives thereof, and an optional deprotection step of the protected chiral amino acids obtained or of derivatives thereof.

7. Process according to claim 6, in which the proton donor is the solvent, in particular selected from primary, secondary or tertiary alcohols with 1 to 8 carbon atoms and in particular selected from methanol, ethanol, n-propanol, n-butanol, isopropanol, sec-butanol, isobutanol and tert-butanol.

8. Process according to claim 6, in which the base is selected from: MHCO₃, M₂CO₃, M′CO₃, MOH, MOAc, R^{c}R^{d}R^{e}N,
M denoting a single-charge cation belonging to the alkali family and selected from the lithium ion Li⁺, sodium ion Na⁺, potassium ion K⁺, caesium ion Cs⁺,
M′ denoting a double-charge cation belonging to the alkaline-earth family and selected from the calcium ion Ca²⁺ and the barium ion Ba²⁺,
R^{c}, R^{d}, R^{e} being selected from H or a carbon chain in particular having 1 to 6 carbon atoms, selected independently of one another.

9. Process according to claim 6, in which the transition metal is selected from rhodium, iridium or palladium.

10. Process according to claim 6, in which the catalyst containing a transition metal comprises [RhCl(C₂H₄)₂]₂, [RhCl(cod)]₂ where cod denotes 1,5-cyclooctadiene, [RhCl(nbd)]₂ where nbd denotes norbornadiene, [RhCl(coe)₂]₂ where coe denotes cyclooctene, [RhCl(CO)₂]₂, [RhOH(cod)]₂, [RhOH(nbd)]₂, [Rh(acac)(C₂H₄)₂]₂ where acac denotes acetylacetonate, [Rh(acac)(coe)₂], [Rh(acac)(cod)], [Rh(cod)₂]BF₄, [Rh(nbd)₂]BF₄, [Rh(cod)₂]PF₆, [Rh(cod)₂]ClO₄, [Rh(cod)₂]OTf where TfO denotes trifluoromethanesulphonate, [Rh(cod)₂]BPh₄.

11. Process according to claim 6, in which the biphosphorus ligand is selected from: (R)-Binap, (S)-Binap, (R)-Difluorphos, (S)-Difluorphos, (R)-Synphos, (S)-Synphos, (R)-MeO-biphep, (S)-MeO-biphep, (R)-Segphos, (S)-Segphos and in particular (S)- or (R)-Difluorphos.

12. Process according to claim 6, in which the organoboron derivative has the following formula: 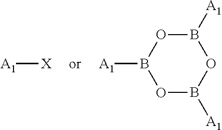
in which:
A₁ is selected from:
1. Aryls having rings with 6 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals with 1 to 15 carbon atoms,
with optionally substituted alkenyl radicals with 1 to 15 carbon atoms,
with optionally substituted alkynyl radicals with 1 to 15 carbon atoms,
with optionally substituted aryls with 6 to 12 carbon atoms,
with optionally substituted aromatic or non-aromatic heterocycles with 2 to 12 carbon atoms,
in which R^{a} and R^{b}, which may be identical or different, represent linear or branched, aromatic or heterocyclic non-aromatic or aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted linear or branched alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted aryls with 6 to 12 carbon atoms,
with optionally substituted aromatic or non-aromatic heterocycles having from 2 to 12 carbon atoms,
2. A₁ is further selected from the heterocycles or the heteroaryls having rings with 2 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
3. A₁ is further selected from linear or branched alkenyls having from 1 to 12 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
4. A₁ is further selected from the linear or branched alkynyls having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
5. A₁ is further selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
X is selected from B(OH)₂, B(OR)₂, BF₃M, B(OR′)₃M,
in which:
R is an alkyl group having from 1 to 14 carbon atoms, (OR)₂ optionally forming a ring between the two oxygen atoms. The groups (OR)₂ in particular originate from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and esters thereof,
or (OR)₂ in particular originates from diacids such as 2,2′-(methylazanediyl)diacetic acid (mida),
R′ is an alkyl group having from 1 to 14 carbon atoms, (OR)₃ optionally forming a ring between two of the oxygen atoms or a dicyclic ring between the three oxygen atoms. The groups (OR)₃ in particular originate from triols such as 2-(hydroxymethyl)-2-methylpropane-1,3-diol,
M represents the lithium ion Li⁺, sodium ion Na⁺, potassium ion K⁺, caesium ion Cs⁺, ammonium ion R^{c}R^{d}R^{e}R^{f}N⁺ where R^{c}, R^{d}, R^{e}, R^{f} are selected from H or a saturated carbon chain in particular having 1 to 6 carbon atoms selected independently of one another,
and in particular A₁-B(OH)₂, A₁-B(OR)₂ or A₁-BF₃K.

13. Process according to claim 6, in which the starting product is a compound of formula: 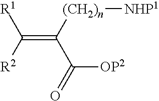
in which:
P¹ is an amine protective group selected from:
COR³, in which R³ represents a linear or branched alkyl, alkenyl, alkynyl group; benzyl, phthalimido (in this case NH is replaced with N) optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
COOR⁴, in which R⁴ represents an alkyl group, more particularly methyl, ethyl, propyl, benzyl, tert-butyl, but also alkenyl, alkynyl, linear or branched, benzyl optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P¹ is in particular selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine,
P² is a carboxylic acid protective group in particular selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkenyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkynyl groups having from 1 to 15 carbon atoms and optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched benzyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched silyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms.
P² is in particular selected from linear or branched alkyl groups having from 1 to 15 carbon atoms, optionally substituted, and in particular methyl, ethyl, isopropyl, tert-butyl and benzyl,
n is equal to 0 or 1,
R¹ and R² are selected, independently of one another, from alkyl or aromatic groups containing from 1 to 10 carbon atoms, provided that at least R¹ or R² is a hydrogen.

14. Process according to claim 6, in which the chiral α- or β-amino acids or derivatives thereof have the formula: 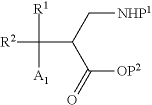
in which:
A₁ is selected from:
1. Aryls having rings with 6 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals with 1 to 15 carbon atoms,
with optionally substituted alkenyl radicals with 1 to 15 carbon atoms,
with optionally substituted alkynyl radicals with 1 to 15 carbon atoms,
with optionally substituted aryls with 6 to 12 carbon atoms,
with optionally substituted aromatic or non-aromatic heterocycles with 2 to 12 carbon atoms,
in which R^{a} and R^{b}, which may be identical or different, represent linear or branched, aromatic or heterocyclic non-aromatic or aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted linear or branched alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted aryls with 6 to 12 carbon atoms,
with optionally substituted aromatic or non-aromatic heterocycles having from 2 to 12 carbon atoms,
2. A₁ is further selected from the heterocycles or the heteroaryls having rings with 2 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
3. A₁ is further selected from linear or branched alkenyls having from 1 to 12 carbon atoms optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
4. A₁ is further selected from the linear or branched alkynyls having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
5. A₁ is further selected from linear or branched alkyl groups having from 1 to 15 carbon atoms optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P¹ is an amine protective group selected from:
COR³, in which R³ represents a linear or branched alkyl, alkenyl, alkynyl group; benzyl, phthalimido (in this case NH is replaced with N) optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
COOR⁴, in which R⁴ represents an alkyl group, more particularly methyl, ethyl, propyl, benzyl, tert-butyl, but also alkenyl, alkynyl, linear or branched, benzyl optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkenyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P¹ is in particular selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine,
P² is a carboxylic acid protective group in particular selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkenyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkynyl groups having from 1 to 15 carbon atoms and optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched benzyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched silyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms.
P² is in particular selected from linear or branched alkyl groups having from 1 to 15 carbon atoms, optionally substituted, and in particular methyl, ethyl, isopropyl, tert-butyl and benzyl,
R¹ and R² are selected, independently of one another, from alkyl or aromatic groups containing from 1 to 10 carbon atoms, provided that at least R¹ or R² is a hydrogen.

15. Process according to claim 6, in which the starting product has the formula: 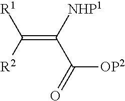
in which:
P¹ is an amine protective group selected from:
COR³, in which R³ represents a linear or branched alkyl, alkenyl, alkynyl group; benzyl, phthalimido (in this case NH is replaced with N) optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
COOR⁴, in which R⁴ represents an alkyl group, more particularly methyl, ethyl, propyl, benzyl, tert-butyl, but also alkenyl, alkynyl, linear or branched, benzyl optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P¹ is in particular selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine,
P² is a carboxylic acid protective group in particular selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkenyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkynyl groups having from 1 to 15 carbon atoms and optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched benzyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched silyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms.
P² is in particular selected from linear or branched alkyl groups having from 1 to 15 carbon atoms, optionally substituted, and in particular methyl, ethyl, isopropyl, tert-butyl and benzyl,
R¹ and R² are selected, independently of one another, from alkyl or aromatic groups containing from 1 to 10 carbon atoms, provided that at least R¹ or R² is a hydrogen,
and is used for preparing a chiral derivative of formula: 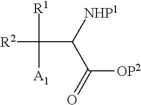
in which:
A₁ is selected from:
1. Aryls having rings with 6 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals with 1 to 15 carbon atoms,
with optionally substituted alkenyl radicals with 1 to 15 carbon atoms,
with optionally substituted alkynyl radicals with 1 to 15 carbon atoms,
with optionally substituted aryls with 6 to 12 carbon atoms,
with optionally substituted aromatic or non-aromatic heterocycles with 2 to 12 carbon atoms,
in which R^{a} and R^{b}, which may be identical or different, represent linear or branched, aromatic or heterocyclic non-aromatic or aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted linear or branched alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted aryls with 6 to 12 carbon atoms,
with optionally substituted aromatic or non-aromatic heterocycles having from 2 to 12 carbon atoms,
2. A₁ is further selected from the heterocycles or the heteroaryls having rings with 2 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
3. A₁ is further selected from linear or branched alkenyls having from 1 to 12 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
4. A₁ is further selected from the linear or branched alkynyls having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
5. A₁ is further selected from linear or branched alkyl groups having from 1 to 15 carbon atoms optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
R¹ and R² have the same meanings as defined above.

16. Process according to claim 12, in which the starting product has the formula: 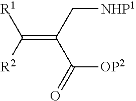
in which:
P¹ is an amine protective group selected from:
COR³, in which R³ represents a linear or branched alkyl, alkenyl, alkynyl group; benzyl, phthalimido (in this case NH is replaced with N) optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
COOR⁴, in which R⁴ represents an alkyl group, more particularly methyl, ethyl, propyl, benzyl, tert-butyl, but also alkenyl, alkynyl, linear or branched, benzyl optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P¹ is in particular selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine,
P² is a carboxylic acid protective group in particular selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkenyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkynyl groups having from 1 to 15 carbon atoms and optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched benzyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched silyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkenyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms.
P² is in particular selected from linear or branched alkyl groups having from 1 to 15 carbon atoms, optionally substituted, and in particular methyl, ethyl, isopropyl, tert-butyl and benzyl,
R¹ and R² are selected, independently of one another, from alkyl or aromatic groups containing from 1 to 10 carbon atoms, provided that at least R¹ or R² is a hydrogen,
and is used for preparing a chiral derivative of formula: 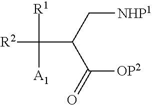
in which:
A₁ is selected from:
1. Aryls having rings with 6 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals with 1 to 15 carbon atoms,
with optionally substituted alkenyl radicals with 1 to 15 carbon atoms,
with optionally substituted alkynyl radicals with 1 to 15 carbon atoms,
with optionally substituted aryls with 6 to 12 carbon atoms,
with optionally substituted aromatic or non-aromatic heterocycles with 2 to 12 carbon atoms,
in which R^{a} and R^{b}, which may be identical or different, represent linear or branched, aromatic or heterocyclic non-aromatic or aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted linear or branched alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted aryls with 6 to 12 carbon atoms,
with optionally substituted aromatic or non-aromatic heterocycles having from 2 to 12 carbon atoms,
2. A₁ is further selected from the heterocycles or the heteroaryls having rings with 2 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
3. A₁ is further selected from linear or branched alkenyls having from 1 to 12 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
4. A₁ is further selected from the linear or branched alkynyls having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
5. A₁ is further selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkenyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
R¹ and R² have the same meanings as defined above.

17. Process according to claim 13, in which P¹ is an amine protective group selected from: (R)-Binap, (S)-Binap, (R)-Difluorphos, (S)-Difluorphos, (R)-Synphos, (S)-Synphos, (R)-MeO-biphep, (S)-MeO-biphep, (R)-Segphos, (S)-Segphos and in particular (S)- or (R)-Difluorphos, and in particular selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine.

18. Process according to claim 13, in which P² is a carboxylic acid protective group selected from: (R)-Binap, (S)-Binap, (R)-Difluorphos, (S)-Difluorphos, (R)-Synphos, (S)-Synphos, (R)-MeO-biphep, (S)-MeO-biphep, (R)-Segphos, (S)-Segphos and in particular (S)- or (R)-Difluorphos, and in particular selected from the methyl, ethyl, propyl, isopropyl, butyl, isobutyl, sec-butyl, tert-butyl, and in particular, isopropyl groups.

19. Process according to claim 6, for preparing a compound of formula: 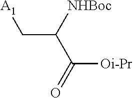
in which A₁ is selected from:
1. Aryls having rings with 6 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals with 1 to 15 carbon atoms,
with optionally substituted alkenyl radicals with 1 to 15 carbon atoms,
with optionally substituted alkynyl radicals with 1 to 15 carbon atoms,
with optionally substituted aryls with 6 to 12 carbon atoms,
with optionally substituted aromatic or non-aromatic heterocycles with 2 to 12 carbon atoms,
in which R^{a} and R^{b}, which may be identical or different, represent linear or branched, aromatic or heterocyclic non-aromatic or aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted linear or branched alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted aryls with 6 to 12 carbon atoms,
with optionally substituted aromatic or non-aromatic heterocycles having from 2 to 12 carbon atoms,
2. A₁ is further selected from the heterocycles or the heteroaryls having rings with 2 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
3. A₁ is further selected from linear or branched alkenyls having from 1 to 12 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
4. A₁ is further selected from the linear or branched alkynyls having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
5. A₁ is further selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
by reaction between a compound of formula: 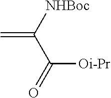
and a boronic acid of formula:
A₁-B(OH)₂
in a medium comprising:
a protic solvent selected from primary, secondary or tertiary alcohols containing from 1 to 8 carbon atoms and in particular selected from methanol, ethanol, n-propanol, n-butanol, isopropanol, sec-butanol, isobutanol and tert-butanol,
a base selected from: MHCO₃, M₂CO₃, MOAc, MOH, M′CO₃, R^{c}R^{d}R^{e}N,
in which
M denotes a single-charge cation belonging to the alkali family and selected from the lithium ion Li⁺, sodium ion Na⁺, potassium ion K⁺, caesium ion Cs⁺,
M′ denotes a double-charge cation belonging to the alkaline-earth family and selected from the calcium ion Ca²⁺ and the barium ion Ba²⁺,
R^{c}, R^{d}, R^{e} being selected from H or a carbon chain in particular having 1 to 6 carbon atoms, selected independently of one another,
[RhCl(C₂H₄)₂]₂, and
a biphosphorus ligand selected from: (R)-Binap, (S)-Binap, (R)-Difluorphos, (S)-Difluorphos, (R)-Synphos, (S)-Synphos, (R)-MeO-biphep, (S)-MeO-biphep, (R)-Segphos, (S)-Segphos.

20. Process according to claim 6, for preparing a compound of formula: 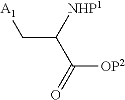
in which
A₁ is a group of formula: 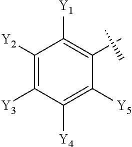
in which Y₁, Y₂, Y₃, Y₄ and Y₅ are selected independently of one another from:
a hydrogen,
an alkyl or aromatic group comprising 1 to 10 carbon atoms,
a halogen,
—CN,
—CO₂Me,
—CF₃,
—COMe,
—NO₂,
—NHAc,
—NHBoc,
—SMe,
—OMe,
—OH,
—OCF₃, and
—NMe₂
or a group selected from: 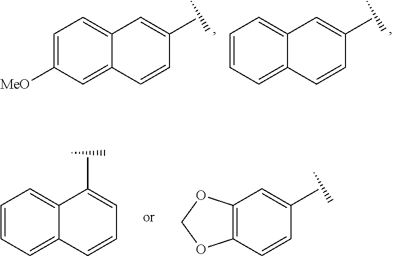
P¹ is an amine protective group selected from:
COR³, in which R³ represents a linear or branched alkyl, alkenyl, alkynyl group; benzyl, phthalimido (in this case NH is replaced with N) optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
COOR⁴, in which R⁴ represents an alkyl group, more particularly methyl, ethyl, propyl, benzyl, tert-butyl, but also alkenyl, alkynyl, linear or branched, benzyl optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P¹ is in particular selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine,
and in particular P¹ is selected from tert-butyloxycarbonyl (Boc), (9H-fluoren-9-yl)methyloxycarbonyl (Fmoc), benzyloxycarbonyl (Cbz or Z), ethyloxycarbonyl (EtOCO), allyloxycarbonyl (Alloc), phthalimido, trihalogenmethylcarbonyl in which the halogen is fluorine, chlorine, bromine or iodine,
P² is selected from the methyl, ethyl, propyl, isopropyl, butyl, isobutyl, sec-butyl, tert-butyl, and in particular, isopropyl groups,
by reaction between a compound of formula: 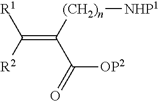
in which R¹=R²=H,
n=0,
in which P² is a carboxylic acid protective group in particular selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkenyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched alkynyl groups having from 1 to 15 carbon atoms and optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched benzyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
P² is further selected from linear or branched silyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms.
P² is in particular selected from linear or branched alkyl groups having from 1 to 15 carbon atoms, optionally substituted, and in particular methyl, ethyl, isopropyl, tert-butyl and benzyl, and a boronic acid, of formula: A₁-B(OH)₂
in which A₁ is selected from:
1. Aryls having rings with 6 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals with 1 to 15 carbon atoms,
with optionally substituted alkenyl radicals with 1 to 15 carbon atoms,
with optionally substituted alkynyl radicals with 1 to 15 carbon atoms,
with optionally substituted aryls with 6 to 12 carbon atoms,
with optionally substituted aromatic or non-aromatic heterocycles with 2 to 12 carbon atoms,
in which R^{a} and R^{b}, which may be identical or different, represent linear or branched, aromatic or heterocyclic non-aromatic or aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted linear or branched alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted aryls with 6 to 12 carbon atoms,
with optionally substituted aromatic or non-aromatic heterocycles having from 2 to 12 carbon atoms,
2. A₁ is further selected from the heterocycles or the heteroaryls having rings with 2 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
3. A₁ is further selected from linear or branched alkenyls having from 1 to 12 carbon atoms optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
4. A₁ is further selected from the linear or branched alkynyls having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
5. A₁ is further selected from linear or branched alkyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in which R^{a} and R^{b}, which may be identical or different, represent linear or branched, aromatic or heterocyclic aromatic or non-aromatic alkyl, alkenyl, alkynyl groups having from 1 to 15 carbon atoms, optionally substituted:
with one or more halogen atoms comprising fluorine, chlorine, bromine or iodine,
with hydroxy, amino or thio radicals optionally protected by ad hoc protective groups,
with —OR^{a}, —NHR^{a}, —NR^{a}R^{b}, —SR^{a}, —OCOR^{a}, —OCONHR^{a}, —OCONR^{a}R^{b}, —CHO, —COR^{a}, —COOH, —CN, —COOR^{a}, —CONHR^{a}, —CONR^{a}R^{b}, —CF₃, —NO₂, —N═C—NHR^{a}, —N═C—NR^{a}R^{b}, —N═C—NH₂, —N═C—NHCOR^{a}, —N═C—NH—COOR^{a}, —N(C═NH)NH₂, —N—(C═NCOR^{a})NHCOR^{b}, —N(C═NCOOR^{a})NHCOOR^{b} radicals,
with optionally substituted alkyl radicals having from 1 to 15 carbon atoms,
with optionally substituted linear or branched alkenyl radicals having from 1 to 15 carbon atoms,
with optionally substituted linear or branched alkynyl radicals having from 1 to 15 carbon atoms,
with optionally substituted linear or branched aryl radicals having from 6 to 12 carbon atoms,
with optionally substituted aromatic or non-aromatic heterocycles having from 2 to 12 carbon atoms,
in a medium comprising:
a protic solvent selected from methanol, ethanol, n-propanol, n-butanol, isopropanol, sec-butanol, isobutanol and tert-butanol,
NaHCO₃,
[RhCl(C₂H₄)₂]₂, and
the biphosphorus ligand (S)- or (R)-Difluorphos.
